(19) United States
(12) Patent Application Publication
(10) Pub. No.: US 2026/0357284 A1
(43) Pub. Date: Nov. 12, 2026

(54) EIF4E KNOCKOUT MAIZE PLANTS FOR VIRAL RESISTANCE

(71) Applicant: LIMAGRAIN EUROPE, SAINT-BEAUZIRE (FR)

(72) Inventors: Stéphane LAFARGE, LA ROCHE BLANCHE (FR); Christophe SALLAUD, BEAUMONT (FR); Jacques ROUSTER, MIREFLEURS (FR)

(21) Appl. No.: 19/099,260

(22) PCT Filed: Jul. 27, 2023
(86) PCT No.: PCT/EP2023/070825

§ 371 Date: Jan. 28, 2025

(30) Foreign Application Priority Data
Jul. 29, 2022 (EP) 22306144.1

(51) Int. Cl. C12N 15/82; C12N 9/22; C12N 15/11

(52) U.S. Cl. CPC C12N 15/8283; C12N 9/226; C12N 15/11; C12N 15/8213; C12N 2310/20

(57) ABSTRACT

The present invention relates to a genetically modified maize plant resistant to at least one maize virus, wherein said maize plant comprises maize cells knocked out for at least one eIF4E gene. The maize virus may be a potyvirus or a machlomovirus. The present invention also relates to a method for obtaining a genetically modified maize plant resistant to at least one maize virus, wherein said method comprises a) knocking out at least one eIF4E gene in a maize cell or maize tissue, to obtain a knocked out maize cell or a knocked out maize tissue and b) regenerating a maize plant from the knocked out maize cell or knocked out maize tissue. The present invention also relates to a vector suitable for knocking out at least one maize eIF4E gene.

### FIELD OF THE INVENTION

The present invention relates to resistance of maize to viral diseases.

### BACKGROUND OF THE INVENTION

Maize can be affected by many viral diseases, in particular caused by several potyviruses, such as Sugarcane Mosaic Virus (SCMV), Maize Dwarf Mosaic Virus (MDMV), Wheat Streak Mosaic Virus (WSMV), which results in combination with MCMV (Maize Chlorotic Mottle Virus (Machlomovirus genus in the Tombusviridae family) to Maize Lethal Necrosis Disease (MLND).

Insecticide treatments are efficient to protect maize culture from MNLD and, until recently, there was few research to find alternatives to these treatments. As insecticide regulations become more stringent in some parts of the world, this leads to increased insect pressure and indirectly to an increase in plant viruses, since insects are important vectors of viruses.

Plant genetic resistance to some viruses does exist in nature. Introgression of this genetic resistance in commercial varieties is an alternative to chemical treatments generating no impact on the environment. Resistance to these viruses can be acquired by example using natural variability, or introgression of QTLs known to confer a resistance to such diseases (see Murithi et al., Frontiers in Genetics, 2021, 12, 767883: 1-17) or by using favorable allele of genes known to be involved in resistance to SCMV for example (see Leng et al. Molecular Plant, 2017, 10:1357-1359).

Natural resistance is not always available in some crops. However, similar resistance can be created by mutagenesis, transgenesis or new breeding techniques.

eIF4E proteins have been used as genetic target to improve genetic resistance of many vegetable crops. eIF4E proteins are indeed known to have a role in virus replication in plants. It was first demonstrated that eIF4E knock-out (KO) vegetable plants did confer a tolerance to several viruses of the potyviridae family. However, the plant phenotype associated with eIF4E knocking-out was shown to be often impaired, such as for example bolting delay, slow growth, affected fertility or even lethality (see Bastet et al., Trends in Plant Science, 2017, 22(5):411-419).

WO2004/057941 discloses a method for imparting virus resistance to plants, said method comprising silencing a gene encoding eIF4E in the plant under conditions effective to impart virus resistance to the plant. Silencing is for example performed using an antisense oligonucleotide complementary to a mRNA encoding eIF4E, in Capsicum and tomato.

There is therefore a need to provide maize plants resistant to viral diseases, such as diseases caused by potyvirus and/or machlomovirus, as an alternative to the use of insecticide treatments.

### SUMMARY

The Inventors have surprisingly found that eIF4E knocking out confers virus resistance to maize, in particular resistance to at least one potyvirus and/or machlomovirus, without affecting the growth, yield and fertility of said maize plants.

These results were unexpected, since eIF4E knocking out in other plants species has been shown to result in bolting delay, slow growth, affected fertility or even lethality.

In a preferred embodiment, eIF4E1 and/or eIF4E2 genes are knocked out to confer virus resistance to maize.

Without being bound by any theory, the Inventors believe that the maize plant knocked out for the eIF4E gene remains viable, because they further comprise eIFiso4E proteins, which may compensate the absence of the eIF4E proteins. eIFiso4E proteins are eukaryotic translation initiation factor 4E isoforms. Surprisingly, virus resistance in the maize plant according to the invention is not circumvented by recruitment of these isoforms by the virus infecting maize.

In a preferred embodiment, eIF4E gene knockout is obtained by gene editing, preferably using CRISPR-Cas.

A first object of the invention is a genetically modified maize cell, comprising at least one knocked out eIF4E gene.

Said eIF4E gene may be eIF4E1 gene or eIF4E2 gene.

The genetically modified maize cell as defined above may comprise a knocked out eIF4E1 gene and a knocked out eIF4E2 gene.

Another object of the invention is a genetically modified maize plant resistant to at least one virus infecting maize or a part of said plant, wherein said plant or said part comprises cells as defined above. Said plant is not obtained by means of an essentially biological process.

Another object of the invention is a genetically modified maize seed resistant to at least one virus infecting maize, wherein said seed comprises cells as defined above.

Another object of the invention is a progeny of a genetically modified maize plant as defined above.

Said virus is preferably a potyvirus or a machlomovirus.

Said potyvirus is preferably selected from the group consisting of Sugarcane Mosaic Virus (SCMV), Maize Dwarf Mosaic Virus (MDMV) and Wheat Streak Mosaic Virus (WSMV).

Said machlomovirus is preferably MCMV (Maize Chlorotic Mottle Virus).

The genetically modified maize plant as defined above, the part of said plant as defined above or the genetically modified maize seed as defined above may be resistant to at least two viruses infecting maize as defined above.

Another object of the invention is a vector suitable for knocking out at least one maize eIF4E gene, wherein said vector comprises:
a) at least one gRNA expression cassette, wherein said gRNA expression cassette comprises a nucleic acid encoding a gRNA under the control of a promoter and wherein said gRNA comprises a region complementary to a target region of said maize eIF4E gene, and
b) optionally, at least one CRISPR-Cas endonuclease expression cassette.

Another object of the invention is a method for obtaining a genetically modified maize plant resistant to at least one virus infecting maize, wherein said method comprises:
a) knocking out at least one eIF4E gene in a maize cell or maize tissue, to obtain a knocked out maize cell or a knocked out maize tissue, and
b) regenerating a maize plant from the knocked out maize cell or knocked out maize tissue.

The step of knocking out at least one eIF4E gene may comprise introducing at least one vector as defined above into said maize cell or maize tissue.

Another object of the invention is a method for identifying a maize plant resistant to at least one virus infecting maize, wherein said method comprises assessing (i) the expression of at least one eIF4E protein and/or (ii) the sequence of at least one eIF4E gene, wherein a maize plant is identified as resistant to at least one maize virus (i) in the absence of expression of said at least one eIF4E protein and/or (ii) in the presence of mutation(s) in the sequence of said at least one eIF4E gene leading to a non-functional eIF4E gene.

### DESCRIPTION OF THE INVENTION

Maize eIF4E Genes and Proteins

The term “eIF4E” refers to Eukaryotic Translation Initiation Factor 4E.

eIF4E protein is a translation initiation factor encoded by the eIF4E gene.

There are two maize eIF4E proteins: protein eIF4E1, which is encoded by an eIF4E1 gene and protein eIF4E2, which is encoded by an eIF4E2 gene.

A reference sequence for native maize protein eIF4E1 is for example sequence SEQ ID NO: 1, also referred to as Zm00001d041682_eIF4E1 in B73 genome version 4 (identified as ZmA188v1aHC012309_eIF4E1 in J1A genome derived from A188). The maize protein eIF4E1 may comprise or consist of a sequence at least 80%, at least 85%, at least 88%, at least 90%, at least 92%, at least 95%, at least 98% or at least 99% identical to sequence SEQ ID NO: 1. The maize protein eIF4E1 preferably comprises or consists of sequence SEQ ID NO: 1.

The eIF4E1 gene is located on chromosome 3, more precisely at positions chr3:133075625 to 133080137 in B73 genome version 4.

The eIF4E1 gene comprises 5 exons. Exon 1 is located in chr3: 133075814 to 133076061 in strain B73. Exon 2 is located in chr3: 133077529 to 133077694 in strain B73.

A reference sequence for native eIF4E1 gene is for example sequence SEQ ID NO: 8.

A reference sequence for native maize protein eIF4E2 is for example sequence SEQ ID NO: 2, also referred to as Zm00001d041973_eIF4E2 in B73 genome version 4, (identified as ZmA188v1aHC012641_eIF4E2 in J1A genome derived from A188).

The maize protein eIF4E2 may comprise or consist of a sequence at least 80%, at least 85%, at least 88%, at least 90%, at least 92%, at least 95%, at least 98% or at least 99% identical to sequence SEQ ID NO: 2. The maize protein eIF4E1 preferably comprises or consists of sequence SEQ ID NO: 2.

The eIF4E2 gene is located on chromosome 3, more precisely at positions chr3:146285064 to 146288191 (reverse strand) in B73 genome version 4.

The eIF4E2 gene comprises 5 exons. Exon 1 is located on chr3: 146288074 to 146287821 in strain B73. Exon 2 is located chr3: 146286386 to 146286221 in strain B73 (reverse strand).

A reference sequence for native eIF4E2 gene is for example sequence SEQ ID NO: 9.

Maize comprises a single copy of the eIF4E1 gene and a single copy of the eIF4E2 gene.

The skilled person knows how to easily localize the eIF4E1 and eIF4E2 genes, as well as the corresponding exons 1 and 2, in a given maize strain, for example by sequence alignment with the sequences of said corresponding genes and exons in strain B73 as defined above.

### Maize Apb1 Qene

The apb1 gene is located on chromosome 3 in maize, more precisely at positions chr3: 3:134550012 to 134554530 in B73 genome version 4.

A reference sequence for the apb1 gene is for example sequence SEQ ID NO: 10.

The apb1 gene encodes protein APB1.

A reference sequence for native maize protein APB1 is sequence SEQ ID NO: 11, also referred to as Zm00001d041711_APB1 in B73 genome version 4.

Apb1 gene is located between eif4e1 and eif4e2 loci. In some strains, regulator sequences in the promoter of the apb1 gene have been identified as responsible for resistance to the Sugarcane Mosaic Virus (SCMV), a potyvirus (see Leng et al. 2017).

### Maize Virus

By “maize virus” or “virus infecting maize”, it is herein meant a virus able to infect maize.

The maize virus as defined above is preferably a potyvirus or a Machlomovirus.

The potyvirus may for example be selected from the group consisting of Sugarcane Mosaic Virus (SCMV), Maize Dwarf Mosaic Virus (MDMV) and Wheat Streak Mosaic Virus (WSMV).

The machlomovirus is for example MCMV (Maize Chlorotic Mottle Virus).

The expression “resistance to at least one maize virus” as used herein thus means resistance to the disease resulting from an infection with said at least one virus infecting maize.

The disease may for example be Maize Lethal Necrosis Disease (MLND), which is caused by the combination of (i) MCMV and (ii) SCMV, MDMV, WSMV or others potyviruses.

### Genetically Modified Maize Cell

The present invention thus relates to a genetically modified maize cell, wherein said cell comprises at least one knocked out eIF4E gene.

As used herein, “maize cell” or “maize plant cell” means a maize cell obtained from or found in a seed, suspension culture, embryo, meristematic region, callus tissue, leave, root, shoot, gametophyte, sporophyte, pollen, or microspore.

The genetically modified maize cell also includes a protoplast, in particular obtained from a tissue as defined above, as well as a cell from or found in a plant cell tissue culture from which plants can be regenerated or plant calli.

The eIF4E gene, which is knocked out, is an endogenous eIF4E gene.

By “endogenous gene”, it is herein meant a gene naturally present in the maize cell.

The eIF4E gene is particularly as defined above.

The eIF4E gene, which is knocked out in the genetically modified maize cell, for example comprises or consists of a sequence having at least 75%, preferably at least 80%, at least 85%, at least 88%, at least 90%, at least 92%, at least 95%, at least 96%, at least 97%, at least 98%, or at least 99%, identity to sequence SEQ ID NO: 8 or SEQ ID NO: 9.

In one embodiment, the eIF4E gene, which is knocked out in the genetically modified maize cell comprises or consists of sequence SEQ ID NO: 8 or SEQ ID NO: 9.

The eIF4E gene may be eIF4E1 gene of sequence SEQ ID NO: 8 or eIF4E2 gene of SEQ ID NO: 9.

The cell as defined above may thus comprise a knocked out eIF4E1 gene and/or a knocked out eIF4E2 gene.

The cell as defined above preferably comprises a knocked out eIF4E1 gene and a knocked out eIF4E2 gene.

By “knocked out gene”, it is herein meant a non-functional gene. A knocked-out gene can no longer express the full length-protein initially encoded by the gene.

In other words, a knocked-out gene can no longer express a functional protein, meaning that full length protein initially encoded by the gene is not translated anymore, but that a non-functional protein can be translated from said knocked-out gene. By “non-functional protein”, it is herein meant a protein lacking at least one function of the functional protein, in particular of the native protein.

The gene may be knocked out by any suitable method well-known by the skilled person, such as using:
a nucleic acid comprising a mutation or a gene to be inserted in the eIF4E gene by homologous recombination,
a site-specific nuclease for introducing a double-stranded break in the eIF4E gene,
a zinc-finger nuclease comprising a DNA binding domain targeting the eIF4E gene and a restriction endonuclease, thereby resulting in double stranded break in the eIF4E gene,
TALEN (Transcription activator-like effector nuclease) comprising a DNA binding domain targeting the eIF4E gene and a nuclease, thereby resulting in double stranded break in the eIF4E gene, and/or
gene editing, for example using CRISPR/cas system, comprising a guide RNA complementary to a target sequence in the eIF4E gene, said guide RNA being complexed with a Cas protein, thereby resulting in a double stranded break in the eIF4E gene and optionally introducing a desired sequence.

In the present application, the term “gene editing” includes insertion, deletion, or substitution of at least one base pair and leads to knock-out, change in the reading frame, specific amino acid change (including STOP codon creation) or a combination thereof. Gene editing involves breaking of DNA strands and DNA damages repair mechanism such as non-homologous end joining or homologous recombination.

When a double stranded break occurs in the eIF4E gene, the cellular DNA repair mechanisms attempts to repair this double stranded break, usually through non-homologous end joining (NHEJ), which results in insertions-deletions (INDELs). INDELs may change the reading-frame and/or introduce non-sense mutation(s), thereby leading to a non-functional eIF4E gene and thus to a disrupted or truncated protein.

Gene knockout is thus different from gene silencing.

Gene silencing is indeed the regulation of gene expression in a cell, to prevent the expression of a given gene. Gene silencing generally reduces the expression of a gene but does not suppress totally (or entirely) its expression into a functional protein, contrary to gene knockout. Most popular gene silencing method is RNAi.

The expression “knocked-out gene” thus does not encompass a gene mutation resulting in a decrease in the level of expression of the protein encoded by said gene.

The expression “knocked-out gene” does not encompass a mutation in the promoter of said gene resulting in a decrease in the level of expression of the protein encoded by said gene.

In the present application, the knock-out can be done by introducing at least one mutation, at least one other gene or sequence and/or at least a double stranded break in any part of the genomic sequence, including 5′ UTR, 3′UTR, introns, exons and promoter region of the eif4e gene.

The knock-out is preferably done by introducing at least one mutation, at least one other gene or sequence and/or at least a double stranded break in any part of the eif4e gene, more preferably inside introns and/or exons.

The maize cells comprising a knocked out eIF4E1 gene thus do not express any eIF4E1 protein.

The maize cells comprising a knocked out eIF4E2 gene thus do not express any eIF4E2 protein.

The maize cells comprising a knocked out eIF4E1 gene and a knocked out eIF4E2 gene thus do not express any eIF4E1 protein, nor any eIF4E2 protein.

The maize cells as defined above may express a non-functional protein from the knocked-out eIF4E gene.

By “non-functional protein encoded by the knocked-out eIF4E gene”, it is herein meant a protein, which is not able to participate in protein synthesis.

Any suitable method may be used to assess if a protein encoded by a knocked-out eIF4E gene is non-functional, such as a yeast complementation test or any known standard methods.

In a yeast complementation test, the yeast is made deficient for the eIF4E protein. Said deficient yeast will not grow if the protein encoded by a knocked-out eIF4E gene is non-functional.

The non-functional protein as defined above encoded by the knocked-out eIF4E gene preferably has a length lower than 150 amino acids, preferably lower than 100 amino acids.

The non-functional protein as defined above may for example comprise or consist of a sequence selected from the group consisting of SEQ ID NO: 12, SEQ ID NO: 13, SEQ ID NO: 14, SEQ ID NO: 15, SEQ ID NO: 16 and SEQ ID NO: 17.

In a preferred embodiment, the non-functional protein (i) has a length lower than 100 amino acids and (ii) comprises or consists of an amino acid sequence having at least 60%, at least 65%, at least 70%, at least 75%, at least 80%, at least 85%, at least 88%, at least 90%, at least 92%, at least 95%, at least 98% or at least 99% identity to sequence SEQ ID NO: 12, SEQ ID NO: 13, SEQ ID NO: 14, SEQ ID NO: 16, or SEQ ID NO: 17.

The maize cell as defined above preferably does not comprise any exogenous nucleic acid encoding a maize eIF4E protein, an eIF4E protein from a plant other than maize and/or a variant thereof.

By “exogenous nucleic acid”, it is herein meant a nucleic acid, which is not naturally present in the maize cell. An exogenous nucleic acid can for example be introduced in a cell by genetic engineering.

In other words, the maize cell as defined above has not been further genetically modified to express a maize eIF4E protein, an eIF4E protein from a plant other than maize, nor a variant thereof.

The maize cell as defined above preferably expresses at least one endogenous eIFiso4E (eukaryotic translation initiation factor isoform 4E) protein. The maize cell as defined above thus preferably expresses protein eIFiso4E1 and/or protein eIFiso4E2.

When part of a maize plant, the maize cells as defined above confer to said plant resistance to at least one maize virus.

In a specific embodiment, the maize cell as defined above further comprises at least one apb1 mutated gene.

The apb1 gene is as defined above.

The term “mutated gene” includes at least one mutation, such as an insertion, deletion and/or substitution, by comparison the wild-type gene, such as the apb1 gene of sequence SEQ ID NO: 10. This at least one mutation can occur in promoter, untranslated region, coding region (exon) and/or or non-coding region (intron) of the gene. Said mutated apb1 gene expression preferably increases the resistance to at least one maize virus conferred by the at least one knocked-out eIF4E gene or leads to another virus infection resistance.

Preferably, the apb1 promoter comprises BoxII motif and three SP1 motifs.

### Genetically Modified Maize Plant, Part of Said Plant or Seed Resistant to at Least One Maize Virus

The present invention particularly relates to a genetically modified maize plant resistant to at least one virus infecting maize, a part thereof or a genetically modified maize seed resistant to at least one virus infecting maize.

The part of the maize plant as defined above may be any part of the maize plant.

As used herein, “maize plant part” means a cell, a protoplast, a tissue or an organ from said maize plant (such as embryo, pollen, ovule, seed, flower, kernel, ears cob, leaf, stalk, stem, root, root tips, anthers, silk, branch, husks or stalk).

As used herein, “plant” means whole plant.

Said plant is not obtained by means of an essentially biological process.

The genetically modified maize plant, the part of said plant or the seed as defined above, comprises genetically modified maize cells as defined above in the section “Genetically modified maize cell”.

Said cells thus comprise at least one knocked out eIF4E gene.

A genetically modified maize seed resistant to at least one virus infecting maize means that the plant grown from said seed is resistant to said at least one virus.

Said maize virus is particularly as defined above.

The genetically modified maize plant or genetically modified maize seed as defined above may for example be resistant to at least one potyvirus (such as SCMV, MDMV or WSMV) and/or at least one Machlomovirus (such as MCMV).

Said genetically modified maize plant or genetically modified maize seed is thus resistant to the disease caused by said at least one virus infecting maize.

Said disease is for example as defined above.

The genetically modified maize plant or genetically modified maize seed as defined above may for example be resistant to MNLD.

The genetically modified maize plant or the genetically modified maize seed as defined above may be resistant to at least two viruses infecting maize, for example to at least three viruses infecting maize or at least four viruses infecting maize.

The genetically modified maize plant or the genetically modified maize seed as defined above may for example be resistant to SCMV, MDMV, WSMV and MCMV.

The genetically modified maize plant or the genetically modified maize seed as defined above may, for example, be resistant to SCMV and MCMV.

The genetically modified maize plant or the genetically modified maize seed as defined above may thus for example be resistant to MNLD.

The maize plant or seed as defined above is preferably an agronomic plant or agronomic seed.

By the expression “agronomic plant or agronomic seed”, it is herein meant a plant or seed suitable for production on a large scale, in particular for human and animal food or for industrial purposes.

In one embodiment, the plant as defined above is regenerated from a genetically modified tissue or cell, wherein said tissue or cell comprises at least one knocked out eIF4E gene as described above. Said plant is for example obtained by the method described below.

In one embodiment, the plant as defined above is regenerated from a genetically modified maize seed as defined above. The present invention thus also relates to the progeny of a genetically modified maize plant as defined above, in particular the progeny of a genetically modified maize plant resistant to at least one virus infecting maize or a part of said plant, wherein said plant is not obtained by means of an essentially biological process and wherein said plant comprises cells comprising at least one knocked out eIF4E gene.

In still another embodiment, the plant is a generational descendant of a plant not obtained by means of an essentially biological process as described hereinabove.

The present invention also relates to the progeny, variants and/or mutants of said regenerated plants, provided that they comprise cells comprising at least one knocked out eIF4E gene.

Resistance to a maize virus may for example be assessed in vivo by monitoring the development (or absence of development) of the maize disease resulting from an infection with said maize virus in the genetically modified maize plant to be assessed. The maize virus is preferably inoculated to the plants, for example by mechanical inoculation. The results are preferably compared to wild-type maize plants placed in the same conditions. A decreased development of the maize viral disease or the absence of development of the maize viral disease in the genetically modified maize plants to be assessed by comparison to wild-type maize plants indicates that the genetically modified maize plant is resistant to said maize virus.

Resistance to a maize virus may also be assessed by virus quantification, for example using Elisa method or qPCR, which allows determining virus presence or absence and/or the quantity of virus present in the genetically modified maize plant to be assessed by comparison to wild-type maize plants.

The ELISA method is particularly able to measure the degree of virus accumulation, for example using antibodies specific for said maize virus. Anti-SCMV, anti-MDMV and anti-MCMV antibodies may for example be used.

The maize seed as defined above may be produced from the maize plant as defined above.

The maize plant as defined above may be produced by growing the maize seed as defined above.

Vector Suitable for Knocking Out at Least One Maize eIF4E Gene

The present invention also relates to a vector suitable for knocking out at least one maize eIF4E gene, for example maize eIF4E1 gene and/or maize eIF4E2 gene.

The vector as defined above may be a plasmid.

The vector as defined above may comprise:
a) at least one CRISPR (Clustered Regularly Interspaced Short Palindromic Repeats)-Cas endonuclease expression cassette and/or
b) at least one gRNA expression cassette.

The vector as defined above preferably comprises:
a) at least one gRNA expression cassette, wherein said gRNA expression cassette comprises a nucleic acid encoding a gRNA under the control of a promoter and wherein said gRNA comprises a region complementary to a target region of the maize eIF4E gene, and
b) optionally, at least one CRISPR-Cas endonuclease expression cassette.
Said at least one CRISPR-Cas endonuclease expression cassette and said at least one gRNA expression cassette may be provided in the same vector or in separate vectors.

The CRISPR-Cas endonuclease expression cassette comprises a nucleic acid encoding a Cas endonuclease under the control of a promoter.

The Cas endonuclease is an enzyme, which uses a gRNA as a guide to recognize and performs a double-stranded break at a specific position in a DNA sequence. The Cas endonuclease generally requires the presence of a Protospacer Adjacent Motif (PAM) sequence in the vicinity of the specific targeted position. The PAM sequence is preferably spaced from the targeted position by at most 20 nucleotides. The PAM sequence can differ depending on the Cas endonuclease.

When the Cas endonuclease requires a PAM sequence, the eIF4E gene comprises a PAM sequence downstream the targeted region.

The Cas endonuclease may be selected from the group consisting of Cas9, Cas12a, Cas12b, C2c1 and C2c2.

The Cas endonuclease is preferably Cas9 (CRISPR-associated protein 9) endonuclease. Cas9 endonuclease may for example comprise or consist of sequence SEQ ID NO: 7.

The promoter of the CRISPR-Cas endonuclease expression cassette may be a constitutive promoter selected from the group consisting of ZmUbi promoter, the 35S promoter or the 19S promoter (Kay et al., 1987), the rice actin promoter (McElroy et al., 1990), the pCRV promoter (Depigny-This et al., 1992), the CsVMV promoter (Verdaguer et al., 1998) and the ubiquitin promoter from rice or sugarcane. The promoter of the CRISPR-Cas endonuclease expression cassette is preferably ZmUbi promoter.

The CRISPR-Cas endonuclease expression cassette preferably comprises a terminator, for example SbHSP.

The gRNA expression cassette comprises a nucleic acid encoding a gRNA under the control of a promoter.

The gRNA comprises:
a) a region complementary to a target region of the eIF4E gene, preferably to a target region in an exon of the eIF4E gene, and
b) a scaffold region, which allows binding to the CRISPR-Cas endonuclease encoded by the CRISPR-Cas endonuclease expression cassette.

The eIF4E gene is particularly as defined above. The target region of the eIF4E gene is preferably located in exon 1 or exon 2 of the eIF4E gene. Exons 1 and 2 of eIF4E1 gene and those of eIF4E2 gene are particularly as defined above in the section “Maize eIF4E genes and proteins”.

The promoter of the gRNA expression cassette may for example be selected from the group consisting of an RNA polymerase III promoter (for example TaU6 promoter, ZmU6 promoter or ZmU3), or an RNA polymerase II promoter, such as a constitutive promoter (for example ZmUbi or TaUbi). The promoter of the gRNA expression cassette is preferably ZmU6 promoter.

The gRNA produced by the gRNA expression cassette is thus able to recognize a target region of the eIF4E gene, so that, when complexed to CRISPR-CAS endonuclease, a double-stranded break in introduced in the target region of the eIF4E gene through the action of the CRISPR-CAS endonuclease.

The gRNA preferably comprises at least 15 nucleotides, preferably at least 17 nucleotides, more preferably at least 18 nucleotides and/or at most 25 nucleotides, preferably at most 24 nucleotides, more preferably at most 23 nucleotides.

The gRNA may for example comprise or consist of sequence SEQ ID NO: 3, SEQ ID NO: 4, SEQ ID NO: 5 or SEQ ID NO: 6.

### Method for Obtaining a Maize Plant Resistant to at Least One Maize Virus

The present invention also relates to a method for obtaining a genetically modified maize plant resistant to at least one maize virus, wherein said method comprises:
a) knocking out at least one eIF4E gene in a maize cell or maize tissue, to obtain a knocked out maize cell or a knocked out maize tissue, and
b) regenerating a maize plant from the knocked out maize cell or knocked out maize tissue.

Said maize virus is particularly as defined above.

Resistance to said at least one maize virus particularly confers resistance to the disease caused by said virus infecting maize.

Said disease is particularly as defined above.

Said maize plant resistant to at least one maize virus is particularly as defined above.

### Step a)

Step a) comprises knocking out at least one eIF4E gene in a maize cell or maize tissue, to obtain a knocked out maize cell or a knocked out maize tissue.

The eIF4E gene is an endogenous eIF4E gene, in particular as defined above.

The eIF4E gene may be knocked out by any suitable method well-known by the skilled person as defined above, such:
homologous recombination with a nucleic acid comprising a mutation or a gene to be inserted in the eIF4E gene,
double-stranded break introduced in the eIF4E gene by a site-specific nuclease, a zinc-finger nuclease comprising a DNA binding domain targeting the eIF4E gene and a restriction endonuclease, a TALEN (Transcription activator-like effector nuclease) comprising a DNA binding domain targeting the eIF4E gene and a nuclease or by gene editing.

The eIF4E gene is preferably knocked out by gene editing, more preferably using CRISPR/Cas system.

The maize cell may be a protoplast.

The maize tissue may be an apical meristem, cotyledon, embryo (preferably an immature embryo), pollen and/or microspores.

The maize tissue is preferably an immature embryo of 10 days after fertilization.

The step of knocking out at least one eIF4E gene preferably comprises introducing into said maize cell or maize tissue at least one nucleic acid of interest, preferably at least one vector suitable for knocking out at least one maize eIF4E gene.

Any method for introducing a nucleic acid in a cell or a tissue may be used, such as transformation or using a haploid inducer line.

Said vector suitable for knocking out at least one maize eIF4E gene is for example as defined above.

When more than one vector is used in step a), the maize cell or maize tissue is preferably transformed with the different vectors at the same time.

Any technique suitable for plant cell or plant tissue transformation may be used, such as biolistic particle delivery, PEG transformation, electroporation or agrobacterium transgene delivery.

For Agrobacterium transgene delivery, the vector is first transferred into Agrobacterium, to obtain a transformed Agrobacterium and the maize cell or maize tissue is then transformed with said transformed Agrobacterium. The Agrobacterium is preferably Agrobacterium tumefaciens.

Transformation with said transformed Agrobacterium for example comprises co-culturing the maize cell or maize tissue with Agrobacterium, for example for at least 5 minutes.

Alternatively to transformation, introduction of the vector can be done by using haploid inducer line and associated technologies.

When using a vector suitable for knocking out at least one maize eIF4E gene as defined above, step a) particularly results in:
the expression of the Cas endonuclease(s) from the CRISPR-Cas endonuclease expression cassette(s) and the production of gRNA(s) from the gRNA expression cassette(s), thereby producing a double strand break in the eIF4E gene in the plant genome, and
double-strand break repair, in particular by non-homologous end joining (NHEJ), leading to insertions and/or deletions in the targeted eIF4E gene.

### Step b)

Step b) comprises regeneration of a maize plant from the knocked out maize cell or knocked out maize tissue.

Regeneration of a plant from a plant cell or plant tissue is well known by the skilled person.

In particular, the maize cell or maize tissue may be placed in a culture medium suitable for maize plant growth.

The regeneration of a maize plant from a maize cell or a maize tissue may comprise:
growing said maize cell or maize tissue to obtain a callus, and
regeneration of shoots from the callus.

The growth of the maize cell into a callus and the regeneration of shoots are carried out in any suitable culture media, in particular comprising plant growth regulators.

The growth of the maize tissue into a callus and the regeneration of shoots from the maize tissue may be carried out in any suitable culture media, in particular comprising plant growth regulators.

The method as defined above may further comprise a subsequent step of assessing whether the eIF4E gene is knocked out in the obtained maize plant.

This subsequent step may for example be performed by assessing the resistance of the obtained maize plant to at least one maize virus, for example as defined above. If the obtained maize plant is resistant to at least one maize virus, contrary to the corresponding non-transformed maize plant, the eIF4E gene is knocked out in the obtained maize plant.

This subsequent step may also be performed by assessing the expression of the protein encoded by the target eIF4E gene. If the cells of the maize plant do not express the protein encoded by the target eIF4E gene, the e14E gene is knocked out in the obtained maize plant.

Detecting the expression of the protein encoded by the eIF4E gene may be performed according to any method well-known by the skilled person, such as western-blot or an immunoassay.

### Method for Identifying a Genetically Modified Maize Plant Resistant to at Least One Virus Infecting Maize

The present invention also relates to a method for identifying a maize plant resistant to at least one maize virus, wherein said method comprises assessing (i) the expression of at least one eIF4E protein and/or (ii) the sequence of at least one eIF4E gene, wherein a maize plant is identified as resistant to at least one maize virus (i) in the absence of expression of said at least one eIF4E protein and/or (ii) in the presence of mutation(s) in the sequence of said at least one eIF4E gene leading to a non-functional eIF4E gene.

The present invention also relates to a method for identifying a maize plant resistant to at least one maize virus, wherein said method comprises assessing (i) the expression of eIF4E1 and eIF4E2 proteins and/or (ii) the sequence of eIF4E1 and eIF4E2 genes, wherein a maize plant is identified as resistant to at least one virus infecting maize (i) in the absence of expression of said eIF4E1 and eIF4E2 proteins and/or in the presence of mutation(s) in the sequence of said eIF4E1 and eIF4E2 gene leading to non-functional eIF4E1 and eIF4E2 genes.

Said maize virus is particularly as defined above.

The eIF4E protein and eIF4E gene are particularly as defined above.

Assessing the expression of an eIF4E protein may be performed by any method well known by the skilled person, such as western-blot or an immunoassay.

Assessing the sequence of at least one eIF4E gene may be performed by any method well known by the skilled person, such as sequencing. The obtained sequence may then be analyzed, in order to identify the possible presence of mutation(s), for example by comparing said sequence with the sequence of the endogenous eIF4E gene, and to analyze if said mutation(s), when present, lead to the non-functional eIF4E gene.

The invention will be further illustrated in the following figures and examples.

### BRIEF DESCRIPTION OF THE SEQUENCES

SEQ ID NO: 1 corresponds to the sequence of a native maize eIF4E1 protein (also referred to as ZmA188v1aHC012309_eIF4E1 in FIG. 2A).

SEQ ID NO: 2 corresponds to the sequence of a native maize eIF4E2 protein.

SEQ ID NO: 3 corresponds to the sequence of gRNA14 (ZmeIF4E_a_SpCas9_gRNA14) targeting a region in exon 1 of eIF4E1.

SEQ ID NO: 4 corresponds to the sequence of gRNA47 (ZmeIF4E_b_SpCas9_gRNA47) targeting a region in exon 1 of eIF4E2.

SEQ ID NO: 5 corresponds to the sequence of gRNA ZmeIF4E_a324&b330_SpCas9_gRNA targeting a region in exon 2 of eIF4E1 and eIF4E2.

SEQ ID NO: 6 corresponds to the sequence of gRNA ZmeIF4E_a367&b373_SpCas9_gRNA targeting a region in exon 2 of eIF4E1 and eIF4E2.

SEQ ID NO: 7 corresponds to the sequence of a Cas9 (CRISPR-associated protein 9) endonuclease.

SEQ ID NO: 8 correspond to genomic sequence of eIF4E1 gene.

SEQ ID NO: 9 corresponds to genomic sequence of eIF4E2 gene.

SEQ ID NO: 10 corresponds to genomic sequence of apb1 gene.

SEQ ID NO: 11 corresponds to the sequence of a native APB1 protein.

SEQ ID NO: 12 corresponds to the sequence of the eIF4E1 protein encoded by mutatype 9_16 (AlleleE1_a_short in FIG. 2A).

SEQ ID NO: 13 corresponds to the sequence of the eIF4E1 protein encoded by mutatype 1030 (AlleleE1_b_short in FIG. 2A).

SEQ ID NO: 14 corresponds to the sequence of the eIF4E1 protein encoded by mutatypes 1519 (AlleleE1_c_short in FIG. 2A).

SEQ ID NO: 15 corresponds to the sequence of the eIF4E2 protein encoded by mutatype 1415 (alleleE2_a in FIG. 2B).

SEQ ID NO: 16 corresponds to the sequence of the eIF4E2 protein encoded by mutatypes 58_77 (AlleleE2_b_short in FIG. 2B).

SEQ ID NO: 17 corresponds to the sequence of the eIF4E2 protein encoded by mutatypes 59_83 or 5974 (AlleleE2_c_short in FIG. 2B).

Sequences SEQ ID NO: 18 to 43 are detailed in Table 1.

TABLE 1

| SEQ ID | |
|---|---|
| NO: | Description |
| 18 | ZmA188v1aHC012309_eIF4E1 |
| | in FIG. 1A |
| 19 | ZmA188v1aHC012309_eIF4E1 |
| | in FIG.1 B |
| 20 | AlleleE1a_MutE1_9_16 (FIG. 1A) |
| 21 | AlleleE1a_MutE1_9_16 (FIG. 1B) |
| 22 | AlleleE1b_MutE1_10_30 (FIG. 1A) |
| 23 | AlleleE1b_MutE1_10_30 (FIG. 1B) |
| 24 | AlleleE1c_MutE1_1_1 (FIG. 1A) |
| 25 | AlleleE1c_MutE1_1_1 (FIG. 1B) |
| 26 | AlleleE1c_MutE1_15_30 (FIG. 1A) |
| 27 | AlleleE1c_MutE1_15_30 (FIG. 1B) |
| 28 | AlleleE1c_MutE1_15_19 (FIG. 1A) |
| 29 | AlleleE1c_MutE1_15_19 (FIG. 1B) |
| 30 | ZmA188v1aHC012641_eIF4E2 |
| | FIG. 1C |
| 31 | ZmA188v1aHC012641_eIF4E2 |
| | FIG. 1D |
| 32 | AlleleE2a_MutE2_14_15 (FIG. 1C) |
| 33 | AlleleE2a_MutE2_14_15 (FIG. 1D) |
| 34 | AlleleE2c_MutE2_59_83 (FIG. 1C) |
| 35 | AlleleE2c_MutE2_59_83 (FIG. 1D) |
| 36 | AlleleE2c_MutE2_59_74 (FIG. 1C) |
| 37 | AlleleE2c_MutE2_59_74 (FIG. 1D) |
| 38 | AlleleE2b_MutE2_58_77 (FIG. 1C) |
| 39 | AlleleE2b_MutE2_58_77 (FIG. 1D) |
| 40 | AlleleE2a_MutE2_13_14_15_16 |
| | (FIG. 1C) |
| 41 | AlleleE2a_MutE2_13_14_15_16 |
| | (FIG. 1D) |
| 42 | Consensus (FIG. 2A) |
| 43 | ZmA188v1aHC012641_eIF4E2 |
| | (FIG. 2B) |

### DESCRIPTION OF THE FIGURES

FIG. 1: Nucleic sequence alignments between reference sequences eIF4E1 and eIF4E2 in J1A lines (public gene IDs are ZmA188v1aHC012309_eIF4E1 and ZmA188v1aHC012641_eIF4E2) and those of the GE edited plants focused on the edited sequences in exon 1 and 2 of eIF4E genes. The first line corresponds to the physical position on J1A map. The final code corresponds to internal reference for the mutation in Exon 1, followed by internal reference for Exon 2 (example “AlleleE1a_MutE1_9_16” correspond to an allele with the mutation with internal reference “9” in Exon 1 of eIF4E1 and mutation with internal reference “16” in Exon 2 of eIF4E1. Some alleles can cumulate more than one mutation AlleleE2c mutE2_13_14_15_16 contains mutations with references “13”, “14” and “15” in Exon 1 and “16” in Exon 2. A: eIF4E1 mutations in exon 1; B: eIF4E1 mutations in exon 2; 0: eIF4E2 mutations in exon 1; D: eIF4E2 mutations in exon 2.

FIG. 2: Protein sequence alignments between reference sequences eIF4E1 and eIF4E2 in J1A lines (public gene IDs are ZmA188v1aHC012309_eIF4E1 and ZmA188v1aHC012641_eIF4E2) and those of the GE edited plants leading to a non-functional eIF4E protein(s). A: eIF4E1 protein sequences of reference and GE plants mutated form; B: eIF4E2 protein sequences of reference and GE plants mutated form.

FIG. 3: Phenotyping of GE plants after viral infection as described in example 4. A: MCMV and SCMV; B: MCMV and MDMV. Set of 7 independent eIF4E GE lines (Plants 1 to 7, see table 2) and Control lines J1A and PA405.

### EXAMPLE: KNOCK-OUT OF MAIZE EIF4E GENES CONFER FULL RESISTANCE TO MAIZE VIRAL DISEASES

### Example 1: Identification of Maize eIF4E Genes

Identification of maize eIF4E sequence has been done by blast method using eIF4E protein sequences of other species. Two eIF4E genes have been identified in maize genome of B73 on chromosome 3, named eIF4E1 and eIF4E2:
Zm00001d041682 as eIF4E1
Zm00001d041973 as eIF4E2.

### Example 2: Design of Guides RNA to Create Knock-Out Mutants of Maize eIF4E1 and eIF4E2 Genes

Design of guides RNA targeting eIF4E1 and/or eIF4E2 has been done using Cas-Designer tool (Pack and al., 2015) allowing us to identify appropriate guides RNA to create mutations in the gene creating a non-functional protein. Four gRNA have been selected in exons 1 or 2:
ZmeIF4E_a_SpCas9_gRNA14/Exon1 of eIF4E1 CGACACCAGGCCTGCCTCCGCGG (SEQ ID NO: 3)
ZmeIF4E_b_SpCas9_gRNA47/Exon1 of eIF4E2 CCGTCCTGCGCACGCTACCGAGG (SEQ ID NO: 4)
ZmeIF4E_a324&b330_SpCas9_gRNA/Exon2 of eIF4E1 and eIF4E2 CAAATAGGGTCTTCCCATTTTGG (SEQ ID NO: 5)
ZmeIF4E_a367&b373_SpCas9_gRNA/Exon2 of eIF4E1 and eIF4E2 TTTTCCTCTTCCACAGCTGATGG (SEQ ID NO: 6)

The three underlined nucleotides in 3′ of the above sequences are PAM sequences.

### Example 3: Production of GE Plants Using CRISPR Cas9 by KO/SDN1 Methodology

### 3.1. Cloning

ZmU6 promoter from Zea mays was used to drive expression of each gRNA described above. The DNA fragment of the 2 gRNA transcription system ZmU6::ZmeIF4E_a324&b330_SpCas9_gRNA and ZmU6::ZmeIF4E_a_SpCas9_gRNA47 was synthetized by GenScript Biotech (The Netherlands) and cloned into pUC57 to obtain the plasmid geBGA_12189. The DNA fragment of the 2 gRNA transcription ZmU6::ZmeIF4E_a_SpCas9_gRNA14 and ZmU6::ZmeIF4E_a367&b373_SpCas9_gRNA was synthetized in the same way to give the plasmid geBGA_12190.

The 4 gRNA transcription units from plasmids geBGA_12189 and geBGA_12190 were cloned via Golden Gate reactions into the binary plasmid pBIOS11975 to obtain the final plasmid pBIOS12487. The T-DNA part of this vector contains three cassettes. One allowing Cas9 endonuclease expression driven by the strong Zea mays ubiquitin promoter (ZmUbi) sequence containing the first intron (Christensen et al. 1992); with a Nos terminator. Another cassette consisting of the ZmUbi promoter and first intron, ZsGreen coding sequence (green fluorescent protein derived from Zoanthus sp.; Matz et al., 1999) and Sac66 terminator sequence from the Arabidopsis thaliana ADGP1 gene (Wang et al., 1998) used for screening purposes. Finally, a selection cassette constituted by a rice actin gene promoter and first intron, the bialaphos resistance (Bar) gene from Streptomyces hygroscopicus (Thompson et al., 1987) and Nos terminator sequence (Depicker et al., 1982) was also included.

The binary vector pBIOS12487 described above was transferred Into Agrobacterium LBA4404.

3.2. Transformation with Agrobacterium

Genetic transformation is done by Agrobacterium tumefaciens, according to the protocol of Ishida et al. (1996, Nat Biotechnol, 14(6):745-50) on J1A, in particular on 10 days after fertilization immature embryos. All the media used are referenced in the reference cited. The transformation begins with a co-culturing phase in which the maize immature embryos are brought into contact for at least 5 minutes with Agrobacterium tumefaciens LBA 4404 containing a binary vector containing the genetic element for editing the target genes. The embryos are then placed on LSAs medium for 3 days in the dark and at 25 °C. A first selection is carried out on the transformed calli: the embryogenic calli are transferred on medium containing phosphinotricin LSD5 5 mg/l and cefotaxime at 250 mg/l (for contamination removal by limiting Agrobacterium tumefaciens). This step is carried out 2 weeks in the dark at 25 °C. The second selection step is performed by transferring the embryos which developed on LSD5 medium, to LSD10 medium (10 mg/l of phosphinotricin) in the presence of cefotaxime, during 3 weeks in the same conditions (temperature and light) as above. The third selection step consists in excising the calli to type I (fragments of 1 to 2 mm) and transferred on LSD 10 medium, at 25 °C in the dark for 3 weeks to 25 °C in the presence of cefotaxime.

The regeneration of the plantlets is carried out by excising the calli of type I which proliferated and transferring them from LSZ on medium in the presence of 5 Mg/I of phosphinotricin to cefotaxime for 2 weeks and to 22 °C and under continuous light.

The plantlets having regenerated are transferred on medium containing 100 mg/l of RM+G2 for 2 weeks to 22 °C and Augmentin and under continuous illumination for the development step. Plants obtained are then transferred to the phytotron for acclimatation.

3.3. Identification of GE Plants Mutated Creating a Stop Codon in eIF4E Proteins

Primers were designed around eIF4E edited sites to produce amplicons that were sequenced to determine sequence modifications. The objective was then to identify deletions, insertions, or substitutions in gene sequences. TO plants were selected when GE mutation(s) in eIF4E gene(s) were able to create a modification of the sequence leading to a non-functional eIF4E protein(s). Alignments were performed between reference sequences (eIF4E1 & eIF4E2) and mutated eIF4E1 & eIF4E2 genes in GE edited plants to select mutations impacting protein sequence.

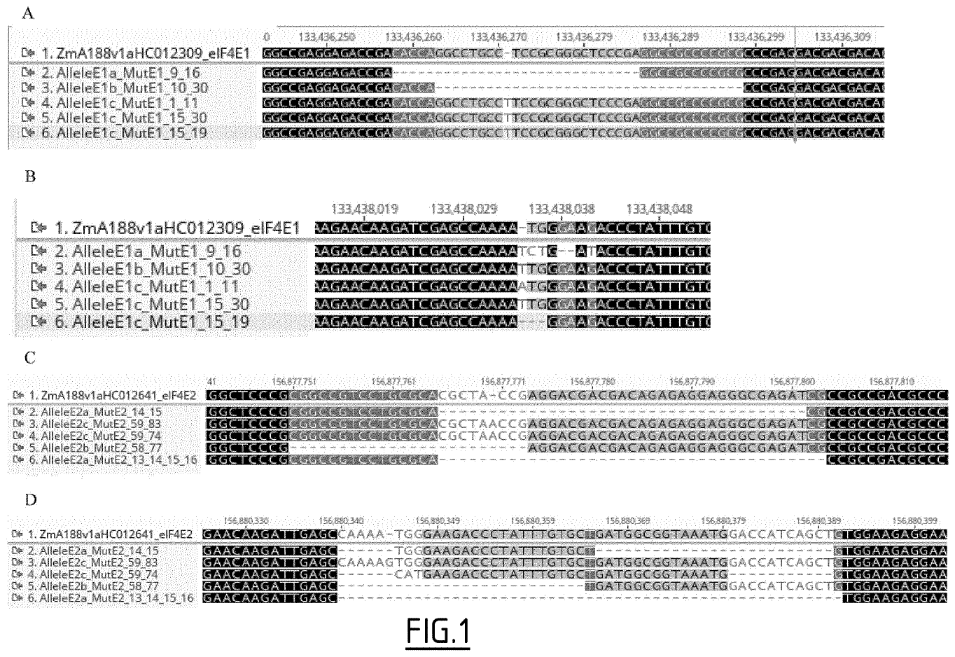

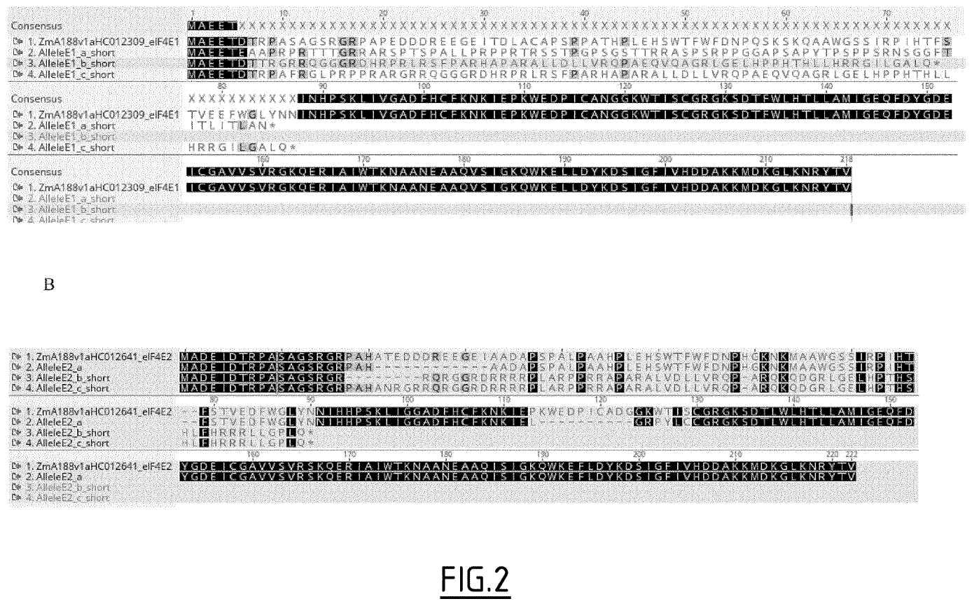

For eIF4E1, we have selected 5 different mutatypes: 9_16; 10_30; 1_11; 15_19; 15_30, which differ from reference sequence at the nucleic level (reference sequence of eIF4E1 in J1A line is public gene ID ZmA188v1 aHC012309_eIF4E1) (see FIG. 1). These nucleic mutatypes give different types of mutated proteins leading to non-functional protein. At protein level, alignments were performed between reference sequence of eIF4E1 in J1A line (public gene ID ZmA188v1aHC012309_eIF4E1) and mutated eIF4E1 proteins (see FIG. 2) showing impact on protein sequence (deletion or creation of an early stop codon). At the end, three different types of mutated proteins have been obtained, named alleleE1a/b/c for eIF4E1.

For eIF4E2, we have selected 5 different mutatypes: 14_15; 59_83; 59_74; 58_77; 12_14_15_16 which differ from reference sequence at the nucleic level (reference sequences eIF4E2 in J1A line is public gene ID ZmA188v1aHC012641_eIF4E2) (see FIG. 1). This nucleic mutatypes give different types of mutated proteins leading to non-functional protein. At protein level, alignments were performed between reference sequences eIF4E2 in J1A line (public gene ID ZmA188v1aHC012641_eIF4E2) and mutated eIF4E2 proteins (see FIG. 2) showing impact on protein sequence (deletion or creation of an early stop codon). At the end, three different types of mutated proteins have been obtained, named alleleE2a/b/c for eIF4E1 and alleleE2a/b/c for eIFE2.

With these alleles for each eIF4E genes we have selected 7 different plants harboring different combinations of eIF4E1/eIF4E2 mutatypes (see Table 2). These T0 plants have been self-pollinated to obtained T1 plants homozygous for the mutation.

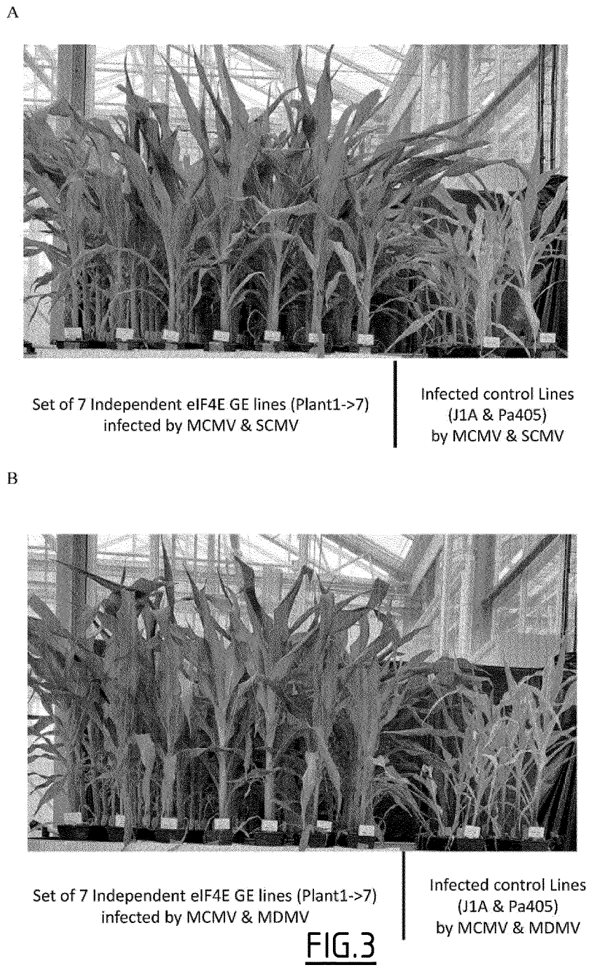

TABLE 2
Description of the truncated protein version corresponding to alleles
mutation for elF4E1 and elF4E2 for the 7 plants of the FIG. 3.

| Plant | Allele | Allele |
|---|---|---|
| Number | ZmelF4E1 | ZmelF4E2 |
| Plant1 | E1c | E2c |
| Plant2 | E1a | E2c |
| Plant3 | E1c | E2a |
| Plant4 | E1c | E2a |
| Plant5 | E1c | E2c |
| Plant6 | E1c | E2c |
| Plant7 | E1b | E2b |

These plants were then submitted to viral infection to evaluate their resistance to different viruses.

### Example 4: Phenotyping of GE Plants after Viral Infection by SCMV, MDMV and/or MCMV

Among the selected GE plants, 7 independent events (Plant 1 to 7, Table 2) (harboring different types of mutations on the two eIF4E genes) were phenotyped in greenhouse to evaluate resistance to 3 different viruses: SCMV & MDMV (Potyviridae) and MCMV (Tombusviridae) alone or by combining MCMV and a potyvirus to mimic Maize Lethal Necrosis Disease.

The maize inbred line J1A was used as a susceptible control and the maize inbred line PA405 as a potyvirus resistant reference. For each event (code Plants 1 to 7, 7 plants were infected, and 2 controls plants were uninfected).

Plants were grown on separate cultivation tables for each modality of viral infection (5 in total) to avoid cross-contamination. Growing conditions were 24 °C Day/19 °C Night with light cycle of 16 h day/8 h night.

13 days after sowing, artificial infection of the plants was performed by mechanic infection of the 2^{nd} or 3^{rd} leaf using fresh infected leaves ground in a phosphate buffer (phosphate 0.03M with 0.2% DIECA, pH 7) containing an abrasive (carborandum, 20 mg/ml phosphate buffer).

The first mosaic symptoms were observed 7 days post infection (DPI) on susceptible plants (J1A). Scoring was performed weekly until 28 DPI. The last observation of mosaic symptoms on infected plants was at 25 dpi. J1A control plants showed mosaic symptoms for all infection modalities with more drastic symptoms under double infection. PA405 plants did not show mosaic symptoms when infected with a potyvirus, as expected, but showed severe symptoms when infected with MCMV alone or in combination with potyvirus. No symptoms were observed on all infected plants from the 7 independent events for all virus infection modalities: SCMV, MDMV, MCMV, SCMV+MCMV, MDMV+MCMV (Table 3 and FIG. 3).

TABLE 3
Results of phenotyping of 7 independent events evaluated
for resistance to 3 different viruses: SCMV & MDMV (Potyviridae)
and MCMV (Tombusviridae) alone or by combining MCMV and
a potyvirus to mimic Maize Lethal Necrosis Disease.

| | | | | MCMV + | MCMV + |
|---|---|---|---|---|---|
| Plant | | | | SCMV | MDMV |
| Number | SCMV | MDMV | MCMV | (FIG. 3, A) | (FIG. 3, B) |
| Plant1 | R | R | R | R | R |
| Plant2 | R | R | R | R | R |
| Plant3 | R | R | R | R | R |
| Plant4 | R | R | R | R | R |
| Plant5 | R | R | R | R | R |
| Plant6 | R | R | R | R | R |
| Plant7 | R | R | R | R | R |
| J1A | S | S | S | S | S |
| PA405 | R | R | S | S | S |

R = Resistant;
S = Susceptible

### REFERENCES

Christensen et al. (1992). Maize polyubiquitin genes: structure, thermal perturbation of expression and transcript splicing, and promoter activity following transfer to protoplasts by electroporation. Plant Mol Biol. 1992 February; 18(4):675-89.
Depicker A, Stachel S, Dhaese P, Zambryski P, Goodman HM. Nopaline synthase: transcript mapping and DNA sequence. J Mol Appl Genet. 1982; 1(6):561-73.
Matz, M., Fradkov, A., Labas, Y. et al. Fluorescent proteins from nonbioluminescent Anthozoa species. Nat Biotechnol 17, 969-973 (1999).
Jeongbin Park, Sangsu Bae, Jin-Soo Kim, Cas-Designer: a web-based tool for choice of CRISPR-Cas9 target sites, Bioinformatics, Volume 31, Issue 24, 15 Dec. 2015, Pages 4014-4016.
Pengfei Leng, Qing Ji, Torben Asp, Ursula K. Frei, Christina R. Ingvardsen, Yongzhong Xing, Bruno Studer, Margaret Redinbaugh, Mark Jones, Priyanka Gajjar, Sisi Liu, Fei Li, Guangtang Pan, Mingliang Xu and Thomas Lubberstedt. Auxin Binding Protein 1 Reinforces Resistance to Sugarcane Mosaic Virus in Maize, Molecular plant, volume 10, October 2017, pages 1357-1360.
Thompson C J, Movva N R, Tizard R, Crameri R, Davies J E, Lauwereys M, Botterman J. Characterization of the herbicide-resistance gene bar from Streptomyces hygroscopicus. EMBO J. 1987 September; 6(9):2519-23.
Wang S M, Lue W L, Yu T S, Long J H, Wang C N, Eimert K, Chen J. Characterization of ADG1, an Arabidopsis locus encoding for ADPG pyrophosphorylase small subunit, demonstrates that the presence of the small subunit is required for large subunit stability. Plant J. 1998 January; 13(1):63-70.
Ishida Y, Saito H, Ohta S, Hiei Y, Komari T, Kumashiro T, High efficiency transformation of maize (Zea mays L.) mediated by Agrobacterium tumefaciens, Nat Biotechnol, 1996 June; 14(6):745-50. doi: 10.1038/nbt0696-74

1. A genetically modified maize cell, comprising at least one knocked out eIF4E gene.

2. The genetically modified maize cell according to claim 1, wherein said eIF4E gene is eIF4E1 gene or eIF4E2 gene.

3. The genetically modified maize cell according to claim 1, wherein said cell comprises a knocked out eIF4E1 gene and a knocked out eIF4E2 gene.

4. A genetically modified maize plant resistant to at least one virus infecting maize or a part of said plant, wherein said plant is not obtained by means of an essentially biological process and wherein said plant or said part comprises cells according to claim 1.

5. A genetically modified maize seed resistant to at least one virus infecting maize, wherein said seed comprises cells according to claim 1.

6. The genetically modified maize plant or part of said plant according to claim 4, wherein said virus is a potyvirus or a machlomovirus.

7. The genetically modified maize plant, the part of said plant or the genetically modified maize seed according to claim 6, wherein said potyvirus is selected from the group consisting of Sugarcane Mosaic Virus (SCMV), Maize Dwarf Mosaic Virus (MDMV) and Wheat Streak Mosaic Virus (WSMV).

8. The genetically modified maize plant, the part of said plant or the genetically modified maize seed according to claim 6, wherein said machlomovirus is MCMV (Maize Chlorotic Mottle Virus).

9. The genetically modified maize plant, the part of said plant or the genetically modified maize seed according to claim 6, which is resistant to at least two viruses infecting maize.

10. A progeny of the genetically modified maize plant according to claim 5.

11. A vector suitable for knocking out at least one maize eIF4E gene, wherein said vector comprises:
a) at least one gRNA expression cassette, wherein said gRNA expression cassette comprises a nucleic acid encoding a gRNA under the control of a promoter and wherein said gRNA comprises a region complementary to a target region of said maize eIF4E gene, and
b) optionally, at least one CRISPR-Cas endonuclease expression cassette.

12. A method for obtaining a genetically modified maize plant resistant to at least one virus infecting maize, wherein said method comprises:
a) knocking out at least one eIF4E gene in a maize cell or maize tissue, to obtain a knocked out maize cell or a knocked out maize tissue, and
b) regenerating a maize plant from the knocked out maize cell or knocked out maize tissue.

13. The method for obtaining a genetically modified maize plant resistant to at least one virus infecting maize according to claim 12, wherein the step of knocking out at least one eIF4E gene comprises introducing at least one vector suitable for knocking out at least one maize eIF4E gene, wherein said vector comprises at least one gRNA expression cassette, wherein said gRNA expression cassette comprises a nucleic acid encoding a gRNA under the control of a promoter and wherein said gRNA comprises a region complementary to a target region of said maize eIF4E gene, and optionally, at least one CRISPR-Cas endonuclease expression cassette, into said maize cell or maize tissue.

14. A method for identifying a maize plant resistant to at least one virus infecting maize, wherein said method comprises assessing (i) the expression of at least one eIF4E protein and/or (ii) the sequence of at least one eIF4E gene, wherein a maize plant is identified as resistant to at least one maize virus (i) in the absence of expression of said at least one eIF4E protein and/or (ii) in the presence of mutation(s) in the sequence of said at least one eIF4E gene leading to a non-functional eIF4E gene.

15. The genetically modified maize seed according to claim 5, wherein said virus is a potyvirus or a machlomovirus.


SEQUENCE LISTING (43 sequences)

SEQ ID NO: 1 - protein, 218 aa, Zea mays
MAEETDTRPASAGSRGRPAPEDDDREEGEITDLACAPSPPATHPLEHSWTFWFDNPQSKSKQAAWGSSIRPIHTFSTVEEFWGLYNNINHPSKLIVGADFHCFKNKIEPKWEDPICANGGKWTISCGRGKSDTFWLHTLLAMIGEQFDYGDEICGAVVSVRGKQERIAIWTKNAANEAAQVSIGKQWKELLDYKDSIGFIVHDDAKKMDKGLKNRYTV

SEQ ID NO: 2 - protein, 220 aa, Zea mays
MADEIDTRPASAGSRGRPAHATEDDDREEGEIAADAPSPALPAAHPLEHSWTFWFDNPHGKNKMAAWGSSIRPIHTFSTVEDFWGLYNNIHHPSKLIGGADFHCFKNKIEPKWEDPICANGGKWTISCGRGKSDTLWLHTLLAMIGEQFDYGDEICGAVVSVRSKQERIAIWTKNAANEAAQISIGKQWKEFLDYKDSIGFIVHDDAKKMDKGLKNRYTV

SEQ ID NO: 3 - DNA, 23 bp, synthetic construct
  source 1..23: mol_type = other DNA
  misc_feature 1..23: note = gRNA
cgacaccaggcctgcctccgcgg

SEQ ID NO: 4 - DNA, 23 bp, synthetic construct
  source 1..23: mol_type = other DNA
  misc_feature 1..23: note = gRNA
ccgtcctgcgcacgctaccgagg

SEQ ID NO: 5 - DNA, 23 bp, synthetic construct
  source 1..23: mol_type = other DNA
  misc_feature 1..23: note = gRNA
caaatagggtcttcccattttgg

SEQ ID NO: 6 - DNA, 23 bp, synthetic construct
  source 1..23: mol_type = other DNA
  misc_feature 1..23: note = gRNA
ttttcctcttccacagctgatgg

SEQ ID NO: 7 - DNA, 4206 bp, synthetic construct
  misc_feature 1..4206: note = SV40 NLS-SpCas9-Nucleoplasmin NLS, maize optimised
  source 1..4206: mol_type = other DNA
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatcatatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaagcgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacaagaggccggcggcaaccaagaaggcgggccaggccaagaagaagaagtga

SEQ ID NO: 8 - DNA, 10520 bp, Zea mays
  source 1..10520: mol_type = genomic DNA; note = eif4e1 genomic sequence with 3'and 5' UTR
atgtttggtttctttagtctaggggctaaagtttagttaggggactaaagtttagtctctaacatgtttggttttaggggctaaaaatagtaagaatataataaatgactcataaaagattaaaatggcctttaacattctcctgctattagtacaattgaactaaatgaggggtaaatgtggaattaatatggtttagtcccttttagcacatatgtgaaggactaaagactaaatcattttagtccatattttagtcctagtgtttgacaaaaaaaagactaaatagaactaaaaactagagactaatctttagttcctctaaccaaacaccacctaatccatccacatgaaagacgatgacacaatacatatctctttaactgccaccaatgccataatacgtgctactcgtgtaggacatgcacacgttggactaggcatgagcaggagcggagcgctcggtgcgcaagccataccttgatttggcagaccaaaagacctttggtcttggcctcgcgtccgcctgctactccttttctccgctgctcgcctaatcactagcttctccaccgttggttgtcgctcgttcgctctacgacgcgcgaggcatggccacacatgtgcctagtcgtgcggccatgccctccatgcttcccctcgatagcgtcgagtgcgttgcggctgttcatactacttcaggcgtctccctagtgtcgagcacttgctagttgactacgtaatgaggtcttggtcaataacccgtgtgattggttgccgcatgttagtagcctggttcggcttggccagcacatgaggagccatgctcatatggccaggctagcttcatacagtagactattgtttggttgtttgcattagtgtattcaggttagatataactcttgtttggttggctgtatcagaacaaagatctgataaaataaataaataaataaataaatctttaaaatttatatgataagtgcactctaattttgttcataaaaatatcaagaacgtagatctgaagttagcttagttattgaacatttcaactaagatatatgataaaaaaacaaaataaaaaaaatatttaggaacacgtatcgaggctctcatatgcatgcatgcagcctgcatccgcagcgacgctcctcagccgtgtacgatgggagcctggctaggaagcaaatcttgcatgtgtggatcctggttgatgcatgcaaggaaccaatcaagttatgctttgaggatccgcggctacaggtgatcggtacatgcaaggaaccaaacatacggaacatgaatgtccttatgggtacgcattccagatctggtttcccattggatgcttagctcttgagatgcttgtctctgtattatatacatgtataaaacttgtgacatctcaaattttgtgtgtccttcaccactgggcatgactataaccacaacactcgaacctagcgctacaaacatagggcccctgctatatcacataccctctccttgctgacgatggcatattgttgaatcagatcatctaaaaggccagagttattacgctcttatagaagttatggttgatagttgtttcaactaaataacttttagcctttttaagccgtgagagattcctgaaaatatgattctatctttgagccaaggtttaaaatagcgggctacagcggcgctatagcctggctatagcgttttgggagaccaaaagctacgatataccattttcacgcgctatatcagctatgagcgctatagctgaaaatagcgccgctaaatacaatagcgctagcgccaaatagctgatgctatttctgttttctagagtgtagggatgaaagtggtaatccgaattgttaggacaaatttaatattttaaagtagatatgtataaaatttgatgttgatcttttcttgtgttatcaagcacattagtacaaatatgaataaaatattacataaaatgttttatgtatgatttggtccctacaacacaaatagttgaaaaaattaccaaatttgtttccgaatccataccgaagtttatatctattatttaagaaaatataggatgaatttaaggcttatcttttatgaatttttactagttggatgttaaaataaagagtacaaatttgtattctatattctatatcctatttattcgcaatcaaagaaaaacgactaaaaaactgattaccgaataaataccgtttccgaccgttttcatccctactagagtgggccaaaaatagagcaatacattgaattcaatagtccatgaaggcccaaaactgaacctaaagtcatctgctgagagggacaaccggacaagcactaagccacatccctgaaacctcaaagaaacggcggcagcggtggcgcgcctgcatgctggtgctgggcacggctacatagggctccagttcgtgaccgcgaggcgaggagaggcaacctgacggaggtgttcaggtgctggcgtggtccttcgtccttgcttagttccttctcttgcttcctagcttcttgctgatagctagctgtccacactattgatctaatgcttgaggacatgggaaagattaagcaagtagatgaaacaattgtgagggcaagatctttgactacattcctttattcccatacaagggttttagcactcatgaggaaattccttggtaaggatctagtgcgtgctggtattacacgcttttctacaacctatttgaatttgaaaagcatgttaggcaacaagaaagagttaggaaagctgtttagatcagatgaactcaatgaaatgggttacttgaaaaaggataagggaaagaacgctagcaaaatagtgaggtctgatacattttgggaaaatgttgattgtgctgtcaatttttttgaaccactagctaatgtacttagaaggatgaatagtgatgtaccagccatgggtttcttatatggttgtttgttggatgcaaagaatgatattgccaaaagatttaacaatgttgaacgttgtttcaaaactatttgggaaattattgataagaggtgggataataagttgaagactcctttacatttggctggttattacttgaatctgtactactattatccaaataaattagagattgagttggatggaacatttagagagggtctagtatcctgtatcacaaagatggttcaagaggttgaaatccaagacaagataatggatgagctcaacatgtaccaggatgggatgggatcgtttggaaaagatattgccacaaggtagaggaggaatgaaaattttaatccaagtgattgaaaatctgacttctcttttcattagcctaatttcacatgtattcattaggccaatctaaatactgatactctactttttatgcagctaaatggtggttgaaccatggaaatagttctccaaatcttagaaagctagcaataaaaattttaggcttaacatgtagctcatcagcatgtgaaaggaattggagtgattttgaaaaggtaagaacagattgtgcaattttcacaattgcttgttactagagcagtagagttgtctcttatgtacttatctaaaatggtcatggtttctataaatctttataggttgactcaaagataaggagcatacttctccatgataggatgagggaccttgtgttcgtcaaatttaactcaaagttgaagcaaaaaaaagagaacaaatcaagagacccaattgagaagcaggttcctgatatcctagaagatagtggcaatgagtggatcactagagttgcatcaattgaacaagaacaagcacaaaacactgaatttgttgaagctacaaatgcacaaggtcagtcatcaaggacaacaatagaaacgggaaaaaggaagagagaaattcagcctagaagaaagacaaggacaaggaagctcattgatattaatgaatgcaatgatgatgaacctaagcttgcttcatcctctgattcagaagaagatgatgcacatgttgacatggatcaagagtcctcaagttctcattcttctgattcggataactccgtgtttgatgaatagaatgcactagtagtagttcacttacactattgcactaataataattttgttatgtaatggatgtctggatcatggtattatgtagctaacttgctacaatgctaccttggtgaactgatgaactacttgtttgctattgtagtaagtagtattttaaaagttggaactattgaattgtttgctgctaaactcttttggcctctgtgttgtttgtttaaggctttaccgctttattaagctgctttccagtctacagctgctcgctgtttggtgtttgctaaggtgtcacaatgtcaactattgattgaatgctgcattgctgcatatgctacaattgttggatggttgtacaactccaactttgactgtgttattgtcagtcttattgactatgtctgtgttattttttacaaatccactatttgctatagctacgctatacctgcgctatagcctcgtagcctaaaaaaatgttgccgctagtttgcatagcccgctattttaaaccttgctttgagctatgagaaaactgccgcgagctggtcgttttggaaaagctaaaatataattctataagagcatgatagcttgtaatacgaataatagaaatatggaaagtctaaggtaaggggtgattctaaattagactagaggaatttatagatttttaagaatctattctgctgttgtacctattttgttgagattccagttttttgacagtgaatctaatcctttgaatattttttttcataaatctacaattcggacagtgttataatgaatttgacgagaagctggtctatcccaaccacctaccatgggcctggcctggcctggcccatttctatgagaagagaggaggagaactgaagggatcaaagggaaaggaaacctagacaagcgaggcggcggccccgcgttctccgtctccgtctccgtctccgtccgcccggcgtaaagaaaagcctctctcaccacagccgccacgccatggccgaggagaccgacaccaggcctgcctccgcgggctcccgaggccgccccgcgcccgaggacgacgacagggaggagggcgagatcaccgacctcgcctgcgctccttccccgcccgccacgcacccgctcgagcactcctggaccttctggttcgacaacccgcagagcaagtccaagcaggccgcctgggggagctccatccgccccatacacaccttctccaccgtcgaggaattctgggggtgcgttcattctcttcagattcgccctccctcgtccttctatcctcttcccctgatcgcatcccactgtttctcttttccgttacaaatcctccgaccgagggccgccgcatgctcatgtaattggtaaattaattcggtgactagtgctagccacttagatgttacgtaaatctatatatgagcccaacctgtcacccttcagttcacggatccgagccgaaactcttgacatatcctagctgctactgttgccttttgattgagtactccttccgtccaaaatgtaaggggttttggctctctttttcccgtctacatccatatccatgttaatgaatctagacacatattcaaaccacatacatgatatataatatttggatcagtaaaaggttagaactccttacaatttgggaccgtggagtaaataagagtaaagttcacttctgatccttaaacttttctgctgtgtcaattctgtacatcaaattttaaagtatgtctcttaagagctcaatctctgtttgagtctctattaagtcctaacaggttgctgtggcccacatgcagtcgttgaggaagaagagagaagggtggatggggtgggggtctaacagaccacaacgactgacatataagccacgacagcccgttaggatgtaatcgaacagagattaagcccttaaaagacatactttaaaagtttatggatcagattgacacagccagacagatttaaggaccggaagtgcactttactcgagtaaataataaattgagcctccaacgtcacacaaaaccttttattctacatagatttgtgaactctttggaggtttcatttagggcatgtaccctgcatattaatgcaccatgccttaagatgtgtcaaagagcttcttcaataaggtgtctcgaaatagtgtcctaaaaattaaaacactacattatttaaagtgtttagaacactaaaaacaaattgaggtccaatagttaagtcctaaatcatgtattttaggaagtaaattacattttttttaataaaaaaatgctaagaataatgtagaaaacaaggatagatcactaacatggggtgtagtacatttgggtcacttcatggtatgtcctatatttgtttgataaaaatttgtaaaatagagtactgttggagccgttttttattatttaagccctatattttaagcccatttatgtgggttatactagtgaagttgtttcgttgattggaaacctgctattgttcacaaccaatatcagttttaccccttctctcctgataacttctctccttctgataagtggtcttttttctagtttcttgatggagcagtggtttgagacatctgctgatctctgcttcattccttttacatcctcgtacagctttaacttccatttattaatgatattccaattccaactaatggatctgtgctttactaggctttacaataacattaatcaccctagcaaattgatcgtgggagctgatttccattgcttcaagaacaagatcgagccaaaatgggaagaccctatttgtgcgaatggtggtaaatggaccatcagctgtggaagaggaaaatctgatacattttggctgcacactgtaagcaattctattttatcagaaagataactttgttttgcacatctctccttaacacgtcctccaccttatattctttcaactgtcctttgtttagctgctggctatgattggtgaacaattcgactatggtgatgaaatttgtggcgcagttgtcagtgtgcgtgggaagcaggaaagaattgctatttggacaaaaaatgctgccaatgaagccgctcaggtatatgcgctgtagtaacagtaacctcttcattggaaacacttgtaccataatttatttattttccttgctaacaaataccatgccttttctcataatattgagttactcaactaacatcttcattggaaacacttgtaccataccttatttagggcgtgtttggaagcaaagtatttcaaaaccatggttttagaatactatggttttgaaatgtcatgccacagctctagtattttttatatcactccaaacaatacttgatttgagacaaaatatctcaaaccataatatttagtatacatgtgcaagaagctagtcatagactttgggttggagtagagtttcaaatactccaaaagtaccatagtatctacaaaactatgttattcaaagcaaagtttgttacaaggtagccaaacacctcttggcaaaaaatgccatggtattctccaataccatggtttcacctcaaaactcaaaaatactttgcttccaaacacccccttattttccttgctaacaaataccatgccttttctcatattttgatcaagtttgagttattcacacataagcatgtattttattgctgtaaccttgccctgtggagcacttcgtaacatatgcatctgtcaaatcaatgtatatagatatttcagggcgtttcattttttttcagtattgttgttagttgttactgtctccttgcggaaccttccactcagctgttttagtcttttgagtaactggtgcaatatcttttgtaatatcaatctaaatatgtaagccggtcggaagccgctttttttgagccgctgcagctatgccacctcacatttgtactgtagaaaaaagcaggtgctactgggaacggcgtccttatctgttgacatttagacaatagttacttctattaaatcaaagtgtgctgggagtagtgccagggtattgccactactttccagtcattgctaatgctgggaactgaaaacctggtgggtattggcaacggtaaaatacatgtggtttttgaacttgttctctccttttcttacgttttcctttccttttcaaactgagagtaaacatccatcagtgcacgtatctcaaatctcagggtctgtttggtaacaatattttttcaaaactatagttttacaatactatacattacaattgtcatatgacaatatcatagttttggccagctccaagaatactagttattgtttggaactttggatacgacattgtaaaacatggtatttaggcaaaagctagccatgctataaaaactttaggttaggttggagtggagtttctaatacactacaaatatcatggttttgaaaatgcagttttcaggaatacatccaaacacctcatggtataaaatactacggtattcatataaaaaccataatattggctataactgcaaaaatacttttactttcaaacaggccctcgtgttactttcattactcaactggaaattcaatgggtattggcctattgggtagtgtggtgcaagtttcgaagaaaacatgtaaatgaaagggaaacatggatggtattggaccaacaaattggctatttccctcgtcctggccatgatacaggaaattgttgcgaggatcagtttttttaacccgtaccagtggattgttcttatgtcttagaaagttataaaatgttgcctcttcagcattgttgtcatttattccatatgtgtaataactaattagactaaacatgccgagcattttcctcctctatcaggtaagcatcgggaagcagtggaaggaacttctggactacaaggactccattggattcattgttcatgtgagcagggcttaagcctccctttgttaatgtcttgtttagtaagctgcaccatttctgactttgcccttttgtgctgcaggacgatgcaaagaagatggacaaaggcctgaagaaccgttacacggtttgatcctgatgctggaatcccagagagttgtagcagtagctggcccaaatgcatgctgggtggttttatctattgctgatgagttgtttaagcaagatccattcctgtattgtactccataagccacttgggtgttgctttgtattgttcactgctgtgttttttctgtggcaccgcatctacagtactagttgcttgtcgttgttcagttgcgcttgaatctgaactaacggagtctgagcaaaacacagcatattcaaacacggcgagcagcaccgtagccaaggcgacaggaactcaggaagtgcggtgaattagatttgttagatataatcaagttgaatttgtatttagactctgatgggcctgtttgtttcagattatataattaggcttaaataatctagatgagaaacaaacagataaattattaagagataaattattaagggggcgtttgaatgcactatacttatagttagttggctaaaaattgttagtggaattagctagctaataaataactaccgaactattaactaatttaccaacaatagctaatagctgaactattagttagggtgtttggatgtttccactaattttagccacgaactattatctcttgtgcattcaaacaccatctaagttagattattgtcgggtaccgtaattaggggtatccccaacactcctaaacacggctggtaaccaccatcattacaagctacagagactgatgggcacgattcaggtcaaggcttcgtctatccaagggacgcgatctcgcctcgtccgagcccagcctcgggcgggaactgtagtcccgaacgaagttacgtctcgcccaagggcctcctcaggcagtgggcgcaccctcggcacgcccgaggcccaactcgggcaagcttcccaactcgggcaagcttcgtcgagaagcaaccttggccaaatcgcctctccaaccgaccgtatcgcagtcgcattcaatacgaggatcgtctgacaccttatcctgacacacgtgcctcagtcggcaggatcgaagtgaccgcagtcacttcaccctttcgctgaccgacctgacaggaaaacagcgccgctcgccccgctccgactactgtaccacccgccagggtgaggctgacaacagccaagttcagcctcgggcgcaacaggaagctccgccttgcccgaccccagggctcggtctcaacctcgaccttggaaggtggtctccgcctcgcccgaccccagggctcggcctcaacctcggcctcggaaggtggtctccgcctcgcccgatcccagggctcggcttcaacctcggcctcgggagga

SEQ ID NO: 9 - DNA, 6996 bp, Zea mays
  source 1..6996: mol_type = genomic DNA; note = eif4e2 genomic sequence with 3' and 5' UTR
gcaactgctgtataattctacccaaatatctgttaaaaaatgtaaacaaaatgattgcaactgctggtgtgttgtaattgcaactgctggtgtggcgtggatgcaactgctgagttactctgatgtgattgcaagtactgaataactctgggaaattttgttaaaaaaatatgattgcaactgttggtctggtgtaattgcaactgctggtctggtgtgattgcaacttctatataactatgtccaaaatctgttaaacaaataatgattgcaactgctgtctggtgtaattgcaactactggtctggtgtgattgcaacttctatgtaactatatccaaaaatctgttaaacaaacaatgattgcaactactgtctggtgtaattgcaactgctgatctggtgtgattgcaacttctatataactatgtccaaaaatctgttaaacaaataatgattgcaactgctgtctggtgtaattgcaactgctggtctggtgtgattgcaacctctatataactatgtccaaaaatctgttaaaacaaacaatgattgcaactgttgtctggtaattgcaactgctggtctagtgtgattgcaacttctatataactatgtccaaaaatttgttaaacaaaacaatgattgcaactgttgtctggtataattgcaactgctgatctggtgtgattgcaacttatttgaagaggttgagccgttcgcggaggagccgttcacggagaaagaggggcggaggcgaggaagtaaaaaagttctgtcagaagcgtgcgggagggacgaagacggcctgggtggggcggttagctcggacctggcgcgtaattcggcctgggtgcattctctatattgaatgcacccaggtgtaatatataaaaacagtgtatatatatatatatatatatatatatatatatatatatatatatatatatatatatatatatatatatatatatatatatatatatatatatatatatatataatatatatatatatatatatatatatatatatatatatatatatatatatatatatatatatatatatatatatatatacacacacacacacacacacaaagctgagacaaaaacaacacctaggcacctaggtgactgacctatggaacatacaggaatacattcgacatcatgttagattatgcttaagcaactcctcagcaaatagagaaccttcaacacactctgcaaggtgctacggaatcggaaagaaccaccctacatgctactactctctgggatcaaaccgtgtaacggttcttcagtcccttgtccatcttctttgcatcgtcctgcagccaaagagggccaagtgagagtaggcacgccttatgagaggaggggtggcttaagcaatgagcactggtactcacatgaacaatgaatccaatggagtccttgtaatccagaaattccttccactgcttcccgatgcttatctgaaaggaaggggggacatgttcagaaccttatgtcaagcatgtcaggaggatgctggcatccaaatgaacttacataagccccccccccccccccccccccccccccaaaaaaaatactgtaaagcatggtatcaaaaaaacaattctcaacatagcaccatggaaatggccaactaccagccggatccccaaccaacaccccccttctgttttcctcgttattctttcgtatatcagggttacatcgataacacagcacgcttatatggaataataactcaaacctgacaatttgcatgtatgataatattggaattgtatcagggaaaaagtatattatttgtaaacatggaaaatatataggttgttgtacaagtattcatcgtgaaaccgaaaactatcataaaagtgtgatgattgcataggagaaaggaaacaagctgtttttaaaagcaacttagaaagagatgcgactgcaggctgcagcacccatacgaccgtacctgagcagcttcattagcagcattttttgtccagatagctattctttcctgcttgctacgcacgctgactactgctccacaaatttcatcaccatagtcgaattgttcgccgatcatagccaacagctacaagaaacagtgtgtaagggtggaggactggaggctataataaggacagatgtcaagaacaaaactcccttttctgataaaataaaactgcttacggtgtgcagccacagcgtgtcagattttcctcttccacagctgatggtccatttaccgccattagcacaaatagggtcttcccattttggctcaatcttgttcttgaagcaatggaagtcagctccaccgatcaacttgctagggtgatggatattattgtagaggctgcgaaatcacagatccattagatggaatatatttatagacgaaagccaaagttggacggggacacaaaaggaccaaaccaaagatagatcagcacatgtctgacacgggattagaccaagagcacttatcacaaggaagagaggaagggttattaaatatcagaccttccaactttcgatcacgcaccacaaccgttaagaaactcgacaggaagtcaagcaagagagccaataacctatgaagctcagttcatcagatcaaatcgaatctcgggttctgaatataaaaaaatatacattacagattttgcgtcatgacagagattgaattttctcatataaactagatccatgaggcgggtgtcactttgacttaataccaactgtagctgtagacaggggcgaatctagggctagggttgaagccccagtcgcagcccaatagcacctatactataccatcattttagcccacaaagtccaactaaaataggtcacaacctccttcctggccgcgccattcagcacctgcgtccggcctccgtccacgccgctagtaccagcgcggcagcgcgggcatccacagcagctagcagtgtggatggccgacacacctaaagcagctacagcagctagcagcgcacaggtgctcagcgaccaatgcaaggcacaagctgcaggcaggctgctgcagcacgccaagcctgcagcacaccgctggctgcccctaagacgtccgccgcatacgctggcgtctccgtcaagtgtgccttatagcccctcttttgatccattcctggatccaccactgactgtagacttgtcaaggcgtgtgtttgagtatgaacaaggactagcaggtaggtctggagatgtttgagaatggaaacataccatgcttacccaataaagatttcatacagtatgtgcatgaatcaaaccgaaagaggtgtaagattgatcgacagctgcttctaaaaaggcaaatgagaaggctaatatctatgatatagcaggggcaaaaacatgtaaaaagtgcagagacagctcgcaacatcagaacaacaggtttgcgtggtttcggggataaatctctaaatggctagaataactaggtgagcaaaaaacatgtgaaaatgaatatctgcttcggataggcgccttgcaaatgaagctaagacagtaactatgctctcagaattgatctttcccgctgtataaacaagtgttagaagctgaatgagagtgctatggattaggcatagtcagaaactaatgcgttaattaatctagagatccaagatccgtaaaccatggcagatttggctcgtatattgtgaatctggacttgtaccagtagttgtgagagtgtgagaggagggaaaggaggttaagaggtgtgcatgtgtcagagagagatagtcggacgcacccccagaagtcctctacggtggagaaggtgtggatgggtcggatggagctcccccaggcggccatcttgttcttgccgtgcgggttgtcgaaccagaaggtccacgagtgctcgagcgggtgcgcggcggggagggcgggcgagggggcgtcggcggcgatctcgccctcctcccggtcgtcgtcctcggtagcgtgcgcaggacggccgcgggagcccgcggaggccggcctcgtgtcaatctcgtcggccatggcagcggcggcggatggggatgggtgcggcgagtcgtgtccaaggaggtttggccgtttggggctcggggctggctgtctggtttttattttttcctttttcttttttttaacggctgacagtctgcgggtctgcggtctttttttaagctaaacatggttccattgatcacttgtattatacacagttgttgttcttccaacttctgtgactactacgattttgtggtcgtcgaaatagtggcgcagttttcgagtactgaggagcaccacgtacaacaacttctagacctgtgggtatctgatcttgagggcccgatgacttcactgatgaaagatattggatgttgaaccggaggtgcgtgtccttcctccttccgctcaactaccaacatggtgcttaccacgtgagtcttgtatgagatatacagtagcagattctggaccggctggtcgctggtggtccggttaagcggcttgaggacgggtgggttgttaggaactttttgagatcctcgaatgctccttgggtatcaggagtccactagaacctgtccaccttttgagcagcttgtacaatagcatgcctctttcgtcgagccatgaaataaaatgactcagcgctgccatgcatccagtaagcttctggaccttttgcgatcgtgatggctccatccgcagtatggattcaatcttcttagggttggcttcaatcccccgatagctgatgatgaaaccaagtatcgaacacacacttgttggggttaagctttcaacggtagcgcctcaggctattgaaaaacttgttggatgtcgtcaatgaagttttctggatcctttgtcttgattacaacatcatcgacataggcttccacacgtttgccccagtggtcggctaagtatgtttggatggcccgctggtaggtcgccctagcattctctaagccgaacgacatggaggtgtaacaaaacaccctgaagggtgtgatgaacgacgtcttctcctgaatcctcctcttgattctcctccactagacccatctggtggtatcccgagtaacagtccaggaaggagagcatggaacaccccgcggtagagttgactacttggtctattctagggagcccgaagggatccttcggatagtgtttgttgagatcagtatagtcgacgcacatgcgccaataaactttattccttttgagtacaagaacaaaattcacaagccactcaagatccgtcacttctctattgaagctagcagctactaggcgagctaactcggctcgtatggcttttctcttatcaggtgtaaagcggcagcttctgtcggatatgttttgcttgcaggtacaccttcagtttgtgctcagtcagttccttgggaactccgggcatgtctgtgagatgccatgcagacacgtctcggttgtcctgcaggaaccggatgagcatgtcttcctatttgccggtgaggctggagctaatggtcgtcgtcttgcgtgcgtcaaggaggccaaggtcggccttcttggtttcttcaactaaccgtatggaagtggccaaggtttcttctttcgccaggatcatgaggtcatcccccggtgatgtgtcagcgttcgtcgtcggtgttcgtggtgccgccaaggggccgatggtgagtgtcatttggatggcccccgggtagcactctgctgctccttggaagtcagcatggatggtgatgacaccctagggctcgggcatcttgagcaccatataagggtagtgggggatggccatgaacttggccagccccggcctcccgttgatggtgttgtagccgcactcgaaccgagccacctcaaagcgtaggaactcagttcggtagttatcaagggtgccaaaggtcacgacatgtagaggtggctgtgggggacagatattccccgggtccactagaaggttggaaggcctcgcgaaagactctaggcccattatttcgcaaggccaccccttcgtgggccaggggaggaatcactggaagagtggatcgacgcaagattggatcaactcgagcccaggcagcccaatggatttaaacgttatccgtagctttaatctgattctcccgcgcagcaccctcagacgttggaactgaacgaggataagtcggcaggataatagggagataagttcagtcggctcactattacttaggagcacatagttatcatacccgcatgtaacgcctcacggtcgagtatataaggcctagggggcaccccatcaagagacatgtcactcattagccatctacactattctctggcatccttcatactagagaatccccctgtaacccaccacataaagatccacaccaggaagtagggtattacacctctctaagcggcccgaacctgtagaaaattgtctatcgtctctcgtgcacctagcacgaaccatcgagctacagtcggttacaccgtcctacccaaaagcacctcgaggggtaacctcaggtgcgcggttgggtcccaaacccaacagctggtgcgccaggtagggggtgtgtcactgatccaagttagctcaatggccatcactcttcagcacaagactgctctccgccccggattcgtgttctgcttcggaaccatctcgttcgtggcagacaaagaagggactctacatcgtatagcggatctgccagagaaaaatccttccttgaaaatctctgagaaagctgaaacaaagcagaaaatagcgcaacctccagcgctccgagcgaagattgcctcctgtagaccaagagcagagatttcattcacccggagaaccccgttgtccacatcatccatagaaaaatggacacagattacaaggaaaaaaaagaaacaaagacccatcaagctgcccttctggtacctctgccctcaaagga

SEQ ID NO: 10 - DNA, 9304 bp, Zea mays
  source 1..9304: mol_type = genomic DNA; note = apb1 genomic sequence 5' and 3'UTR
gaagaggtactctttcaatcatagcaagatagtcacccctgggtcggcgtattcccgccaccttgaagggtccttcccagttgggggagagttgcagagcccttcttggtttggtacttgccgtaggactaggtcccgaccctaggttgccatctgcgtttgatgagggagtcctcgtcctcgtgctgtagccatttctgcatggacctgtcagaagtctagacccatggggagtccagaagggtttccgggggaaggcaggcttcggccccatagaccggggagcacggggtccccctgggggtccatctagttgtccccacctaagtaggagcctctagcgaagacatccttcaggtccccctcgggtgactgatactcgaggtcccgttcagctacgaccacctcaccgtcgtcgaccttttctttgccaggtcggcgatgaggtggggagccgtccctggaagactgctcgcgccgctcgctgacgcatttcgcgaggtcaatgatcttgcgacactctgcggcgttgtggcgaccgttggggtgcacagggcatgagccgctgttacccctctgcgaccgtgggcgtttgttgcggtcgccccggcccccggtcgcggctgcaacaactagagcgatggaccgcgacctctggtagtcgcggcccttcttctttttcttcttaccgtcccgggggacggcacccgagccacccgactgggcagccccggtttgtggagccgagtgccatgcacggccctcggcggctctggcgcttttgtcggccagagcgaagagtgtggggacagtctccacgtcatgcgtggccaacttctccaacattttctcatcacgtactccctgtcggaaggccgtgatgatggaagcatcggaaatgcaaggtatagtacctcgcaccttggtgaagcgggagatgaacgtccggagagtctccccgggctcctgcctcaccgcatggaggtgggcctccacaccgtgctgctggtaagcgctggcgaagtttgcaacgaaccgcgcgcagagctcttcctaggagtagattgaccctggtgcgaggttcatgagccaagtccaggcaggaccagacaaggcgacatgaaaatatgttgccatcacagcggtgtttccaccggctgctatgatagcggtgacatacacctgcaggaacttcgacggatttgacgttccgtcgtacttttccggcaggtgcggccggaacttgggtggccatgacgccgcacggagatgatccgcgagagcggcgcagtccacatcggccaaggggacacccgcttgggatcgggtacccattggggcctgcggtgctaccgcatcgaagtcttgattgaggttgcagccctcgatgttttggcggcgctcgcgcgccctttccaaagagacccgagcgtcctctcctgcacgcctgcgattgagttctgcccgaaggtcgttggtttgtgcacccctcactgagggtgagtgcacagacgtagacgcctcatgttggcgccgggatgacggtggcctcgacctggttgaggtagaatgtgccatgccgagcagtcggtcgacgtcatcccgccactgcttcatggccctcagtgaggccgtggagcttggagggtgtcgcagcaactccctggctactgacaacgctcccggagccgccctcgacggagttcaggatgtctgcgcagctgtatgctgccgtgcagcgtgcacagcagcagtgctcgacactgggcagttgggaacctgtggtgtggaagaaacaacttcttcctccacaaggaaatccgttgatgtctcggcgcggtgctccacgatttgcaccatcatgctgagcgaggatgcaagcaaaaacctaatgcctagccccctacctggcgcgccaaatgtcggagagggaattctccggctgggtggcggagtgcacctgccctaaatcctaagatgaggaaggggcttaagcgttttgcctgtcctgctaagcgaacatcaagcacatgaacacgcgagagtttagagtggttcgggctgccggagcgtaacaccctactccactgtatgttggattgctgtggaagcttgagaattgagagtctaagttgggtctaaaaattgagcttgtgttgtaacgttgcatgcctccccttttatagctaaaggggggcatgtacaagggtactgagcctcgacatgtgggcccaggaacatagcggatgcagtacttggagctactaatatttgctgctggggcaatcttcttgtgccctgacacctgagatctgcgtagcctcggcgtgcaggggaagcttctcgtataagggatgatgagcacagtgcaccgtgcagcacggtcgcactgtttcgacaatggactggataggtgagccgtctgcagggtggccctggcgccgcctgccagcggagtggacatgacgcaataaatgctgagggggcacatcacctgtcagcagggtggacaggcggcgcgtcctttccgtaataaatgcagagtccacgcggtctaggggccttacgtcaggctccgcccgctggcttattcacgggcaacgagccacgtggccgcatcgggtctccgcctgagtggggagcgcatgcgcagagtaaggcccgaccacgtgttagtaccggacccttgctcatgccagggtttcccctgtcccgggacctcgttgcggcccagacctttctcgaagagatctggaccccatccaagggacccgacatgcttactcgggagtcccgggccataatcgggggtccaggttgtgcgtacaggggtccgactcgactgatacatcctggatgtatcatctttccttgccacatggtgccccttgagctgcccatgtggtggggtcgagcgctgttcaccgtgtggctagggaacggcgtacgggcaccgcgtcttcatactgtagtaaggggtacccctgattcagggtaccgacaataattatgtaaactccttgagtaattggtgtctcatgtgtgatttctccatggcgttgcttcataaggatcacaaacatatttgtctcaccttttgaagcaaacataaatcaaaccctgtgacttgtaccatatcaccatatatgagttcaaatcatggcttcaagtcaccttactgatgcatcaacatgttataactcttcataactgattagttcaccgacttagtgcaagtactctcttcttcaccttaaccatggtacttcggtccacaagccgtcgcttggccttcaccttcgcttagttcctcgaagccctttccttgctatcttcaccctatcaaaccatctttgagtcacattatattgatcatccattgagagaatcatttcttcgatattgtgaaccttgcttgaatgtcatctagatatgctaagatcaatcaagctctagtttgattctcataaaagcatatatggactaatagtaatatggccaagccaattcacgatttctcatatcttattcactttggcttgaccaatcctcttaattacttcaacctctattatgatcatatttatgcatagtggtttctaaggtcttgtccatatattcaaaccaatatagagaccgtattatatccatttgcattgtctcactggttatttaaccttgtgttgaacctttattcactgatcattatacactattcaaatatgttcatcatactgaattttctataatataaaatagtgttttgcacgaccataaatggttagagacgttataccgttcttgttgccttgtttatgtcctagcaagattttcgttgataagagaggagagccataaacaggtcgtccggcctggcaagccgaggtttgtcctgcgtggtgcggcttgtctctgggccggctgctcccacaagctatctgagccgctgctccatttgatcggacagtcgacaccgtccgagcggcgatcaaaaccaccttcgacgactacgccgaaaccctagcctctatctcattccactccgacattcacgtgcagctgtcgggagcaggcaatggcgccggatctaagcgaactcgccgccgccgctccagcccgtggcgcctacctcgccggcgtcggtgtcgcggtcctcctcgctgcctccttcctcccagtagccgagtcgtcctgcgtgcgaggtgcaccctcaccctgccgagctttttgccactgagaccagcactgctaagtgctagtggggtttgggcttaactggcgtgttaaatagcaggctgggtcttgttttggtcgttagggagtgggggaaggttgaggggaattgggcttagtggctcggtttgcattgggactaattggggtagtttaggttaacaatcaaattagggtggtttggtgtgggctccaatagtttcagcccatgggccccccaagcccgcgcctccgcctccgcctccgctgccggccgtcgcaacgactccaactccacccaagtccactacaggcctacagcctactcaccgggttactactacagttcgcaactacatccagattccaggctaatgctaatccttaggttggagatgggttggaaacagattgaacttgcggtgacataaatggattggtttggtttggcttggattctgcaagttcacacgcttaattcaaagtatatctgggtggattcagattgatttacagcacaacaattcccatctgccacctcaaagaatggattcaccatttgccacttcgtttttttttactgccattggacatgcgaaatggccatgctgcccataatcctaattgattatgtgtagttagcactcattgctcttcaacctgcaaaaatcaagcatagtgccatgctgtagtttgccgctctagatctacatgaaatggatcccaacccaaaatacagcctatcatctcaattcacgcatagccaccactcaagcaaagtaaattctgaacttgtttttaagtggccaaatcgatctccgtgagacccattacaacagcaaccaacttattttgactcacttggagcacaacggtagtaggggttgggctgcgtgctggagcagcagtggtagtagtggtgtcttaggccttctgcttctgccaagctcaactctgcacccaagaaggtgactgggtctgactgggaggagaagcagttgtaatagtcgcttttgcagtcttctgcttctgccgagctcaacttgccaccagagaagaaggttactgttctgtgggggagcatcgcagtctgctggtcatgccagctaagaacagagctgcatggcctgtctttggccgtcagagcgtggaagcactgaaggttctggcagcattgacttctgtgtcatcatcgtacagtggtagaaaaactaagggaagttgaatggaccaaacattaagatcttggtcaattggggtaaggggcaatatcgactaaaaagagtaaattagcaggaaaagcaaattggcaatgttaaacatggcagatggggagatctgatttcgtaaaaaaactctccctccctttcccctccctcttcattctcttctcgaaattcatgaaacttctaaaattgttgtttgaccttacccacaattttggtgtgaatgagcagtatttctggatataataattcgactggggcagttacatccccccctccctcaaacacacaatattcacaatagcagtgaccatatttctttttctacaattgtagcagatctgattagtgtatctgatattattatgtatacagataactcattggtgagagacataagccaaatgccgcaaagcagctatgggattgaaggattgtcacatataacagttgctggtgcgctcaatcatgggatgaaggaggtaatgataaaccctagggccacatcgtgtttcccaaacaatatgggaatgaacaattgaagtcaaagataagttgaatgtaatcgtgtattttcatgtcttgtatgtgcaggtctggtttctgtggacactgtagatagttactgaaccttgatgcatgatagtttttcacttctatttgaactaaaatttggactgtaacgttcaattggtgtgctatgaaatcaaagtacttaattatgtgtagatatgtttgatagagtcatattcaaatagtctattgacagtgtagatactgttatgcttccttcttgcttcgtcatgaaatgttttcctgcaggtggaagtgtggcttcagacaataagtccaggtcaaaggacgccaatccacaggcattcctgtgaagaagttttcactgtcctcaaagggaagggtacgctcttgatgggatcaagctcactaaagtacccagggcagccacaggaaattcctttctttcagaataccacattttcaatccctgtaaatgatccacaccaggtaaaggaaatgagttattacttatatgcatttttactttttctgatacattagatgcctcatatcctggttatatttaactgtaacttggccagatttcaacatgtaaatttattttggtgggtaggaaattgatctttttgttaatgtatacatcatctttctcttgcaggtttggaattctgacgagcacgaagatttgcaagttcttgtgatcatatcgagaccgcctgctaagatgtgagcatgcctaactttaacatcgtgtttcattttttttctcaaacatgtaatagagctgcacatcattatattaagcagaggaaaaagagatgagtcttaacaactcatacaacccttaaggccatgttcggttctaccccaatccatatggattggattgagggggtttcaatccctagtaagtcaaaatctctccaaatccgtatcaatcccctccaatctatatggattgaaaataaccgaacaagccctaaaggggcaaacaccgcactaggacttaccacacactaatacacaaccaacaccaaggaggaggaatcatggccatagactacagagttgtttttatatttctcattatgctatcaaaactaggtgactccctcaaacacatgcatttctatgtttccagatccaccaagccactaaaactaacaacgagtttaagcccttacgagattgcttagctgctgtgcagacatcaccattcagtaaagatgttgtcccttaacccctgaacatactgctgcagcccaaacaactttagggcagactacaccacacagtccttgcaaatacacaggaggtgagcaaatgttgtatgttctcctcttcttgatcacacaatggacagctctcatgatgctcaagtccccagtgagctaacccgtctgcagtccaacactgatttagagcagctagccaaatgaaaaacttgcatcggaggtgcagcccaaaacttccggatttctttccatgggtcaaaaccaacagccccgaacacccgacaaagaaactacaataagcagatttggttgagaattctcctgtagtggatggtgatcaaatgtgccagtaggtcgtgtattccctggtgaagatcaatttctgctataatatcccatagatggtggtattccatcaaggctgtagctgacagccccctgatgtaccatacccaagcattattgtccaatgcatcctgcaccattcttcttttgattgctctctttgccacatgtgtgtaaagtggagcgggatcaaacgcagatattccctgaaaccatttatcactccaaaaaagttttcggctgtcattgcctaacacagaataagctgaaatgcagaacatggacatagaattacagtgaaccttgtagagagtattcccttatgtttccatcttgttcaactcagtaccatactaagttgacaagacttgattggagaatttgctacctatactgctgtgatttattgatttaccatttttttctgtaattgtccaacattgctagaatagatatcatagtttcatgcacgtgtcatgggcacttttgttatctatgtcagtagcatcttttatgctctagtgttgaccctagcaatattgataatacagcaactgagtaatccacatacgcgtttcttttctccccctgtttgcaattgcatgtcagaaaacgcacataatcatatctgcaatcaacatttttctttctgagatccaatgacatcctcgttgcagatttttatatgatgattggagcatgcctcatacagccgcggtactgaaattccccttcgtctgggatgaggactgcttcgaagcagcaaaagacgaactctaggtcacaagtgtttcctgcaatttatctgcttcatccatgatcctgctggtgctggactactacaattctcagcactagttgtaataaagccagtgcgcttttcatgtataattctgtattgtggctcgcgaaaataaaatttggcaacggtttatgaacttatgcgcttgtggagttgatccaatgttcaccgtttcaagatttttgcattattactgtgctccaggtttcgagcagaagcagtactgccttcatccatgaacttggggtttgtttgatagcccccgtaacagatttcgggcagaatttaggagcttggaaacagtcttagtatgaaaccgagagtggtttctaggatgtaattggatcgaatacggacggatatcactgaaactatatttgttttcatatttttatttggatttgaatttgaatacatatggccttggatacaaatataaatatggccgatgcaaatattgataatcataagtcgaatacggaattaatcgaacacagataacgtcgggtagcagattttttgtcagatatcatataaagaccgttccacacatatcatggttattgtaatcattagtcactttttgagtgacattttattttttaggattatatttggacatatattgtggctctataaaattttatgaatttctaagtatttgagtattattaattttttatgcagaaaatcattaaaatacatttaaatttataaaatattctaatgtcgatcgaaaattgcaaataaattaccaagactagcaaacattctataaaaagataatctcaagtcttcacatgaaaatgataaagtgaaatattgattatgtcgtaacttggatacttagaatgatttcacgtgtaactcatgcaacaagtggaagtgaaatgaaaaaaatgttggtatcttgtggatcttatggcccaaattttatgaggattata

SEQ ID NO: 11 - protein, 201 aa, Zea mays
  source 1..201: note = APB1 amino acid sequence
MAPDLSELAAAAPARGAYLAGVGVAVLLAASFLPVAESSCVRDNSLVRDISQMPQSSYGIEGLSHITVAGALNHGMKEVEVWLQTISPGQRTPIHRHSCEEVFTVLKGKGTLLMGSSSLKYPGQPQEIPFFQNTTFSIPVNDPHQVWNSDEHEDLQVLVIISRPPAKIFLYDDWSMPHTAAVLKFPFVWDEDCFEAAKDEL

SEQ ID NO: 12 - protein, 84 aa, synthetic construct
  source 1..84: note = AlleleE1_a_short MutE1_9_16
MAEETEAAPRPRTTTGRRARSPTSPALLPRPPRTRSSTPGPSGSTTRRASPSRPPGGAPSAPYTPSPPSRNSGGFTITLITLAN

SEQ ID NO: 13 - protein, 74 aa, synthetic construct
  source 1..74: note = AlleleE1_b_short MutE1_10_30_
MAEETDTTRGRRQGGGRDHRPRLRSFPARHAPARALLDLLVRQPAEQVQAGRLGELHPPHTHLLHRRGILGALQ

SEQ ID NO: 14 - protein, 86 aa, synthetic construct
  source 1..86: note = AlleleE1_c_short MutE1_15_19_
MAEETDTRPAFRGLPRPPRARGRRQGGGRDHRPRLRSFPARHAPARALLDLLVRQPAEQVQAGRLGELHPPHTHLLHRRGILGALQ

SEQ ID NO: 15 - protein, 198 aa, synthetic construct
  source 1..198: note = AlleleE2_a MutE2_14_15 / 56_66
MADEIDTRPASAGSRGRPAHAADAPSPALPAAHPLEHSWTFWFDNPHGKNKMAAWGSSIRPIHTFSTVEDFWGLYNNIHHPSKLIGGADFHCFKNKIELGRPYLCCGRGKSDTLWLHTLLAMIGEQFDYGDEICGAVVSVRSKQERIAIWTKNAANEAAQISIGKQWKEFLDYKDSIGFIVHDDAKKMDKGLKNRYTV

SEQ ID NO: 16 - protein, 80 aa, unidentified
  source 1..80: note = AlleleE2_b_short MutE2_58_77
MADEIDTRPASAGSRGRRQRGGRDRRRRPLARPPRRAPARALVDLLVRQPARQKQDGRLGELHPTHSHLFHRRRLLGPLQ

SEQ ID NO: 17 - protein, 88 aa, synthetic construct
  source 1..88: note = AlleleE2_c_short MutE2_59_83 / 59_74
MADEIDTRPASAGSRGRPAHANRGRRQRGGRDRRRRPLARPPRRAPARALVDLLVRQPARQKQDGRLGELHPTHSHLFHRRRLLGPLQ

SEQ ID NO: 18 - DNA, 71 bp, Zea mays
  source 1..71: mol_type = genomic DNA; note = ZmA188v1aHC012309_eIF4E1 (fig 1A)
ggccgaggagaccgacaccaggcctgcctccgcgggctcccgaggccgccccgcgcccgaggacgacgaca

SEQ ID NO: 19 - DNA, 39 bp, Zea mays
  source 1..39: mol_type = genomic DNA; note = ZmA188v1aHC012309_eIF4E1 (fig 1 B)
aagaacaagatcgagccaaaatgggaagaccctatttgt

SEQ ID NO: 20 - DNA, 43 bp, synthetic construct
  source 1..43: mol_type = other DNA; note = AlleleE1a_MutE1_9_16 (fig 1 A)
ggccgaggagaccgaggccgccccgcgcccgaggacgacgaca

SEQ ID NO: 21 - DNA, 38 bp, synthetic construct
  source 1..38: mol_type = other DNA; note = AlleleE1a_MutE1_9_16 (fig 1 B)
aagaacaagatcgagccaaaatctgataccctatttgt

SEQ ID NO: 22 - DNA, 36 bp, synthetic construct
  source 1..36: mol_type = other DNA; note = AlleleE1b_MutE1_10_30 (fig 1 A)
ggccgaggagaccgacaccacccgaggacgacgaca

SEQ ID NO: 23 - DNA, 40 bp, synthetic construct
  source 1..40: mol_type = other DNA; note = AlleleE1b_MutE1_10_30 (fig 1 B)
aagaacaagatcgagccaaaattgggaagaccctatttgt

SEQ ID NO: 24 - DNA, 72 bp, synthetic construct
  source 1..72: mol_type = other DNA; note = AlleleE1c_MutE1_1_11 (fig 1 A)
ggccgaggagaccgacaccaggcctgccttccgcgggctcccgaggccgccccgcgcccgaggacgacgaca

SEQ ID NO: 25 - DNA, 40 bp, synthetic construct
  source 1..40: mol_type = other DNA; note = AlleleE1c_MutE1_1_11 (fig 1 B)
aagaacaagatcgagccaaaaatgggaagaccctatttgt

SEQ ID NO: 26 - DNA, 72 bp, synthetic construct; identical to SEQ ID NO: 24
  source 1..72: mol_type = other DNA; note = AlleleE1c_MutE1_15_30 (fig 1 A)

SEQ ID NO: 27 - DNA, 40 bp, synthetic construct; identical to SEQ ID NO: 23
  source 1..40: mol_type = other DNA; note = AlleleE1c_MutE1_15_30 (fig 1 B)

SEQ ID NO: 28 - DNA, 72 bp, synthetic construct; identical to SEQ ID NO: 24
  source 1..72: mol_type = other DNA; note = AlleleE1c_MutE1_15_19 (fig 1 A)

SEQ ID NO: 29 - DNA, 37 bp, synthetic construct
  source 1..37: mol_type = other DNA; note = AlleleE1c_MutE1_15_19 (fig 1 B)
aagaacaagatcgagccaaaaggaagaccctatttgt

SEQ ID NO: 30 - DNA, 73 bp, Zea mays
  source 1..73: mol_type = genomic DNA; note = ZmA188v1aHC012641_eIF4E2 (fig 1C)
ggctcccgcggccgtcctgcgcacgctaccgaggacgacgacagagaggagggcgagatcgccgccgacgccc

SEQ ID NO: 31 - DNA, 77 bp, Zea mays
  source 1..77: mol_type = genomic DNA; note = ZmA188v1aHC012641_eIF4E2 (fig 1D)
gaacaagattgagccaaaatgggaagaccctatttgtgctgatggcggtaaatggaccatcagctgtggaagaggaa

SEQ ID NO: 32 - DNA, 37 bp, synthetic construct
  source 1..37: mol_type = other DNA; note = AlleleE2a_MutE2_14_15 (fig 1 C)
ggctcccgcggccgtcctgcgcacgccgccgacgccc

SEQ ID NO: 33 - DNA, 47 bp, synthetic construct
  source 1..47: mol_type = other DNA; note = AlleleE2a_MutE2_14_15 (fig 1 D)
gaacaagattgagctgggaagaccctatttgtgctgtggaagaggaa

SEQ ID NO: 34 - DNA, 74 bp, synthetic construct
  source 1..74: mol_type = other DNA; note = AlleleE2c_MutE2_59_83 (fig 1 C)
ggctcccgcggccgtcctgcgcacgctaaccgaggacgacgacagagaggagggcgagatcgccgccgacgccc

SEQ ID NO: 35 - DNA, 78 bp, synthetic construct
  source 1..78: mol_type = other DNA; note = AlleleE2c_MutE2_59_83 (fig 1 D)
gaacaagattgagccaaaagtgggaagaccctatttgtgctgatggcggtaaatggaccatcagctgtggaagaggaa

SEQ ID NO: 36 - DNA, 74 bp, synthetic construct; identical to SEQ ID NO: 34
  source 1..74: mol_type = other DNA; note = AlleleE2c_MutE2_59_74 (fig 1 C)

SEQ ID NO: 37 - DNA, 61 bp, synthetic construct
  source 1..61: mol_type = other DNA; note = AlleleE2c_MutE2_59_74 (fig 1 D)
gaacaagattgagccatgaagaccctatttgtgctgatggcggtaaatggtggaagaggaa

SEQ ID NO: 38 - DNA, 50 bp, synthetic construct
  source 1..50: mol_type = other DNA; note = AlleleE2b_MutE2_58_77 (fig 1 C)
ggctcccgaggacgacgacagagaggagggcgagatcgccgccgacgccc

SEQ ID NO: 39 - DNA, 52 bp, synthetic construct
  source 1..52: mol_type = other DNA; note = AlleleE2b_MutE2_58_77 (fig 1 D)
gaacaagattgagctgatggcggtaaatggaccatcagctgtggaagaggaa

SEQ ID NO: 40 - DNA, 35 bp, synthetic construct
  source 1..35: mol_type = other DNA; note = AlleleE2a_MutE2_13_14_15_16 (fig 1 C)
ggctcccgcggccgtcctgcgcaccgccgacgccc

SEQ ID NO: 41 - DNA, 25 bp, synthetic construct
  source 1..25: mol_type = other DNA; note = AlleleE2a_MutE2_13_14_15_16 (fig 1 D)
gaacaagattgagctggaagaggaa

SEQ ID NO: 42 - protein, 218 aa, synthetic construct
  source 1..218: note = Consensus (Fig2A)
  VARIANT 6..87: note = X can be any amino acid or be deleted
MAEETXXXXXXXXXXXXXXXXXXXXXXXXXXXXXXXXXXXXXXXXXXXXXXXXXXXXXXXXXXXXXXXXXXXXXXXXXXXXXXXXXXINHPSKLIVGADFHCFKNKIEPKWEDPICANGGKWTISCGRGKSDTFWLHTLLAMIGEQFDYGDEICGAVVSVRGKQERIAIWTKNAANEAAQVSIGKQWKELLDYKDSIGFIVHDDAKKMDKGLKNRYTV

SEQ ID NO: 43 - protein, 220 aa, Zea mays
  source 1..220: note = ZmA188v1aHC012641_eIF4E2 (Fig 2B)
MADEIDTRPASAGSRGRPAHATEDDDREEGEIAADAPSPALPAAHPLEHSWTFWFDNPHGKNKMAAWGSSIRPIHTFSTVEDFWGLYNNIHHPSKLIGGADFHCFKNKIEPKWEDPICADGGKWTISCGRGKSDTLWLHTLLAMIGEQFDYGDEICGAVVSVRSKQERIAIWTKNAANEAAQISIGKQWKEFLDYKDSIGFIVHDDAKKMDKGLKNRYTV